(19) United States
(12) Patent Application Publication
(10) Pub. No.: US 2011/0201577 A1
(43) Pub. Date: Aug. 18, 2011

(54) PROCESSES AND INTERMEDIATES FOR THE PREPARATION OF OSELTAMIVIR AND ANALOGS THEREOF

(75) Inventors: Tomas Hudlicky, St. Catharines (CA); Bradford Sullivan, St. Catharines (CA)

(73) Assignee: BROCK UNIVERSITY, ST. CATHARINES, ON (CA)

(21) Appl. No.: 12/992,024

(22) PCT Filed: May 12, 2009
(86) PCT No.: PCT/CA2009/000622

§ 371 Date: Apr. 7, 2011

Related U.S. Application Data
(60) Provisional application No. 61/052,435, filed on May 12, 2008.

(51) Int. Cl. A61K 31/655; C07D 317/46; A61K 31/36; C07D 491/056; A61K 31/396; C07C 229/48; A61K 31/215; C07C 309/77; A61K 31/255; C07C 247/14; C07D 498/04; A61K 31/424; A61P 31/16

(52) U.S. Cl. 514/150; 549/439; 514/465; 548/961; 514/463; 560/125; 514/529; 558/57; 514/517; 552/1; 548/217; 514/375

(57) ABSTRACT

The present application relates to processes for the preparation of oseltamivir and the H₃PO₄ salt of oseltamivir, Tamiflu®. The application further relates to novel intermediate compounds and to pharmaceutical compositions containing said compounds. The application further relates to a method of using the novel intermediates to treat or prevent influenza.

### FIELD OF THE APPLICATION

The present application relates to a process for the manufacture of novel intermediates useful for the preparation of oseltamivir, and oseltamivir phosphate (Tamiflu®) from readily available precursors and processes to prepare oseltamivir, and oseltamivir phosphate.

### BACKGROUND OF THE APPLICATION

The possibility of a major influenza pandemic (especially the avian H5N1 influenza) continues to be a serious health concern. The development of effective antiviral medicines is hampered by the exceptionally high mutation rates of influenza virus. Therefore, in order to be successful, new drugs should target the molecular mechanisms specific to the proliferation of the virus. The mechanism of infection involves the protein neuraminidase (NA), essential to viral replication. NA is responsible for the glycosidic cleavage of sialic acid (1) (in Scheme 1) from a glycoprotein of a host cell in a process that liberates the virion from the infected cell [(a) Russell, R. J.; Haire, L. F.; Stevens, D. J.; Collins, P. J.; Lin, Y. P.; Blackburn, G. M.; Hay, A. J.; Gamblin, S. J.; Skehel, J. J. Nature 2006, 443, 45; (b) Colman, P. M.; Varghese, J. N.; Laver, W. G. Nature 1983, 303, 41]. The NA protein active site appears to be conserved in many strains of the influenza A and B virus. Therefore, an efficient inhibitor of the NA protein could provide a broad-spectrum anti-influenza drug. Two compounds have been found most effective in mimicking the oxonium intermediate of sialic acid glycolysis, (i.e. structure (2) in Scheme 1), and hence acting as excellent inhibitors of NA: zanamivir (3) and oseltamivir phosphate (5), Tamiflu® (also shown in Scheme 1). Tamiflu® appears to be superior to zanamivir because it is orally active and serves as prodrug, the active form of which is the corresponding carboxylic acid. It also has a superior bioavailability and is active at nanomolar levels. For the most recent reviews of Tamiflu activity, supply problems, and its syntheses see: Shibasaki, M.; Kanai, M. Eur. J. Org. Chem. 2008, ASAP web edition; (b) Farina, V.; Brown, J. D. Angew. Chem. Int. Ed. 2006, 45, 7330.

Oseltamivir is not a complex molecule yet its practical synthesis on a scale large enough to guard against an influenza pandemic presents a formidable challenge. Stockpiles of Tamiflu® and similar agents are currently the focus of many governments worldwide.

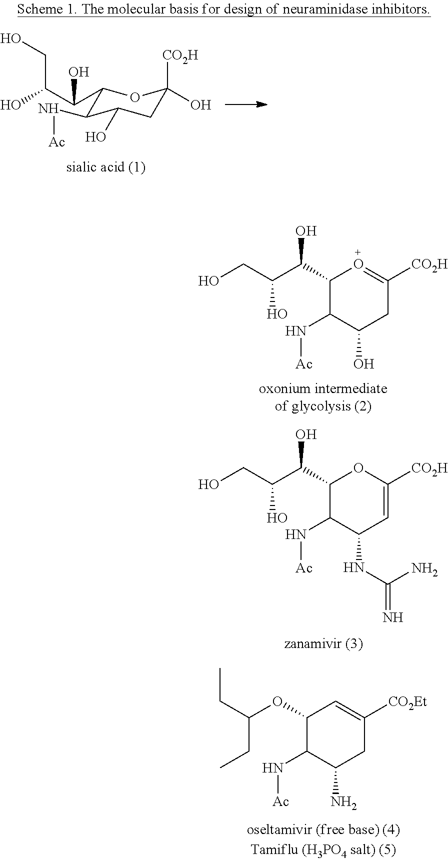

### SUMMARY OF THE APPLICATION

It has been found that the readily available compounds such as bromobenzene and benzoate esters can be used as starting materials in processes for the preparation of oseltamivir and analogs thereof.

The present application therefore includes a first process for the preparation of oseltamivir and analogs thereof of the formula I:

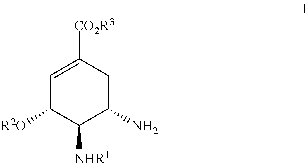

wherein R¹ is C₁₋₆acyl;
R² is C₁₋₆alkyl; and
R³ is C₁₋₆alkyl, said process comprising:
(i) converting a halobenzene of the formula II, wherein X is halo, to the diol of the formula III, wherein X is halo, by toluene dioxygenase mediated oxidation,

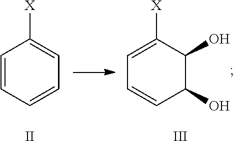

(ii) protecting the hydroxyl groups of the diol of formula III, followed by aziridination to provide a compound of the formula IV, wherein X is halo, R¹ is C₁₋₆acyl and PG¹ and PG² are suitable protecting groups that are optionally linked to form, together with the oxygen atoms to which they are attached, an optionally C₁₋₆alkyl-substituted 5- or 6-membered ring,

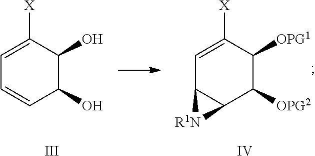

(iii) opening the aziridine ring in the compound of the formula IV with an alcohol of the formula R²—OH, wherein R² is C₁₋₆alkyl, followed by palladium catalyzed carbonylation to provide a compound of the formula V, wherein R¹ is C₁₋₆acyl, R² is C₁₋₆alkyl, R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

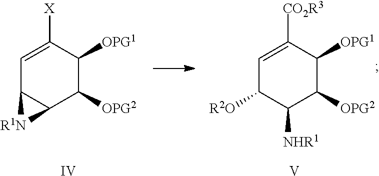

(iv) deprotecting the compound of the formula V and selectively converting the hydroxyl adjacent to the NHR¹ group to a leaving group (LG) to provide a compound of the formula VI, wherein R¹ is C₁₋₆acyl, R² is C₁₋₆alkyl, R³ is C₁₋₆alkyl and LG is a suitable leaving group,

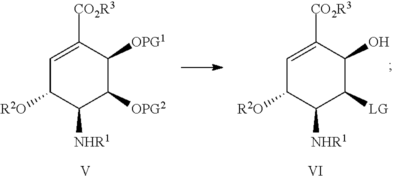

(v) displacing the leaving group in the compound of formula VI with an azide group to provide a compound of the formula VII, wherein R¹ is C₁₋₆acyl, R² is C₁₋₆alkyl and R³ is C₁₋₆alkyl,

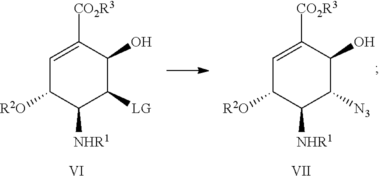

and
(vi) conversion of the hydroxyl group in the compound of the formula VII to a group removable by reduction and treating the resulting compound under reductive conditions to remove the group and converting the azide group to an amino group to provide a compound of formula I,

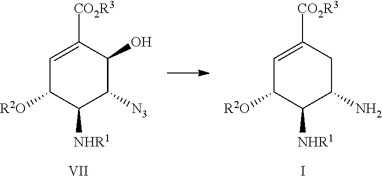

wherein, in the compounds of the formulae kW, one or more hydrogens in R¹, R² and/or R³ is/are optionally replaced with F.

In another embodiment, the compound of formula I is converted to its pharmaceutically acceptable salts. Conversion to the H₃PO₄ salt provides Tamiflu® and analogs thereof.

In another aspect of the application there is provided a second process for the preparation of oseltamivir and analogs thereof of the formula I:

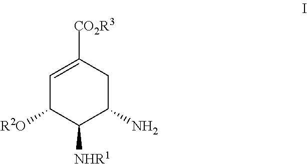

wherein R¹ is C₁₋₆acyl;
R² is C₁₋₆alkyl; and
R³ is C₁₋₆alkyl,
said process comprising:
(i) protecting the hydroxyl groups of a compound of formula III, wherein X is halo, as the di(C₁₋₆)alkyl ketal followed by aziridination to provide a compound of the formula VIII, wherein X is halo, R⁴ and R⁵ are independently, C₁₋₆alkyl and R⁶ is C₁₋₆acyl,

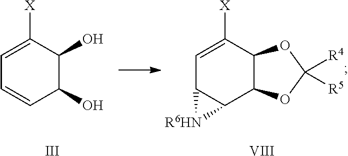

(ii) ring opening the aziridine ring of the compound of formula VIII with PG³-NH₂, where PG³ is a suitable protecting group, followed by palladium catalyzed carbonylation to provide a compound of the formula IX, wherein R³, R⁴ and R⁵ are, independently, C₁₋₆alkyl, R⁶ is C₁₋₆acyl and PG³ is a suitable protecting group,

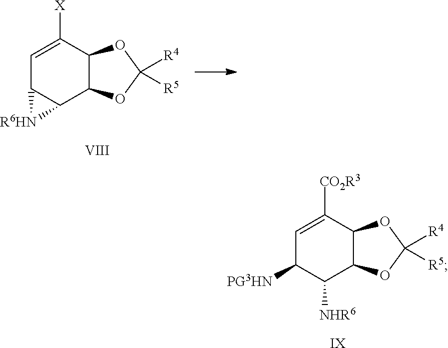

(iii) regioselective ring-opening of the cyclic ketal in the compound of formula IX and reduction to provide a compound of the formula X, wherein R³, R⁴ and R⁵ are, independently, C₁₋₆alkyl, R⁶ is C₁₋₆acyl and PG³ is a suitable protecting group,

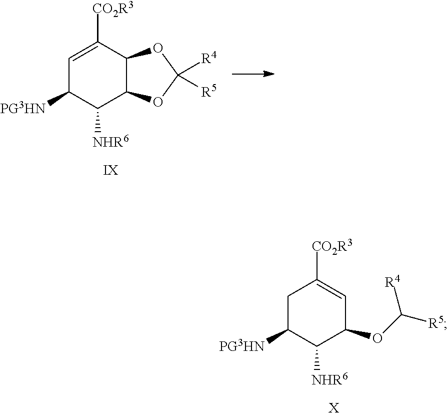

or, removing the cyclic ketal in the compound of formula IX followed by rearranging and eliminating one hydroxy group to provide a compound of the formula XI, wherein R³ is C₁₋₆alkyl, R⁶ is C₁₋₆acyl and PG³ is a suitable protecting group followed by alkylating the remaining hydroxy group with a compound of the formula R²-LG, wherein R² is C₁₋₆alkyl and LG is a suitable leaving group, to provide a compound of the formula XII, wherein R³ is C₁₋₆alkyl, R⁶ is C₁₋₆acyl and PG³ is a suitable protecting group,

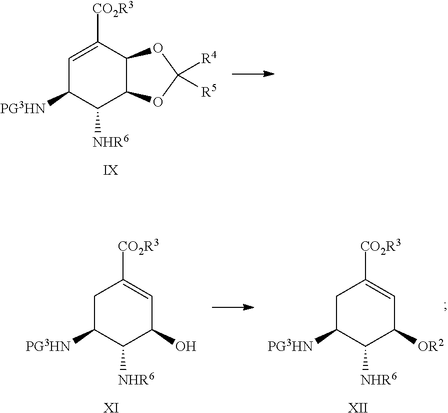

(iv) removing the PG³ of the compound of the formula X or XII to provide a compound of the formula I,

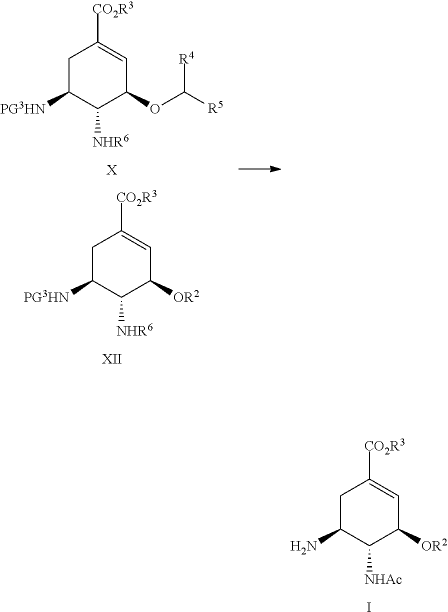

wherein, in the compounds of the formulae I, III and VIII-XII, one or more hydrogens in R¹, R², R³, R⁴, R⁵, and/or R⁶ is/are optionally replaced with F.

In another embodiment, the compound of the formula I is converted to its pharmaceutically acceptable salts. Conversion to the H₃PO₄ salt provides Tamiflu® and analogs thereof.

In another aspect, the present application also includes an alternate process for the preparation of compounds of formula IX

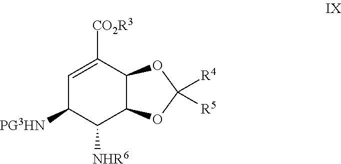

wherein R³, R⁴ and R⁵ are, independently, C₁₋₆alkyl, R⁶ is C₁₋₆acyl and PG³ is a suitable protecting group, said process comprising:
(i) converting a benzoate ester of formula XIII, wherein R³ is C₁₋₆alkyl, to a compound of the formula XIV, wherein R³ is C₁₋₆alkyl, by toluene dioxygenase mediated oxidation,

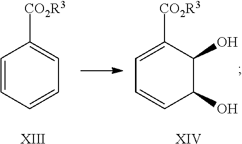

(ii) protecting the hydroxyl groups of the compound of formula XIV as the di(C₁₋₆alkyl) ketal followed by regio- and stereoselective nitroso Diels-Alder cycloaddition to the conjugated diene to provide a compound of the formula XV, wherein R³, R⁴ and R⁵ are, independently, C₁₋₆alkyl and R⁶ is C₁₋₆acyl,

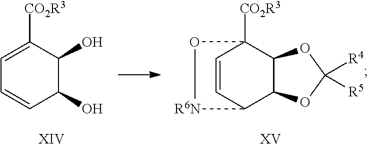

and
(iii) displacing the oxazine bridge of the compound of formula XV with PG³NH₂, wherein PG³ is a suitable protecting group, under palladium catalyzed conditions followed by reduction to provide a compound of the formula IX, wherein R³, R⁴ and R⁵ are, independently, C₁₋₆alkyl and R⁶ is C₁₋₆acyl and PG³ is a suitable protecting group,

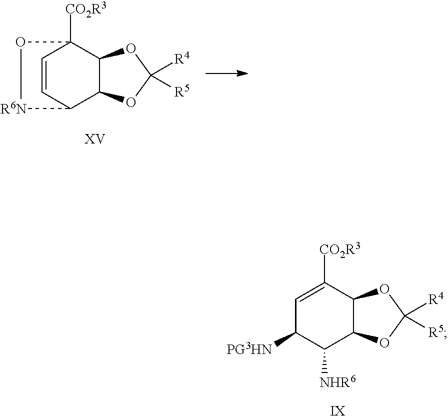

or,
ring opening of the oxazine bridge of the compound of formula XV to yield the compound of formula XVI, followed by converting the hydroxy group in the compound of formula XVI to a leaving group and allylic displacement of the leaving group with PG³NH₂ or azide, wherein PG³ is a suitable protecting group, to provide, after reduction of the azide and protection of the resulting amine with PG³, a compound of the formula IX, wherein R³, R⁴ and R⁵ are, independently, C₁₋₆alkyl and R⁶ is C₁₋₆acyl and PG³ is a suitable protecting group,

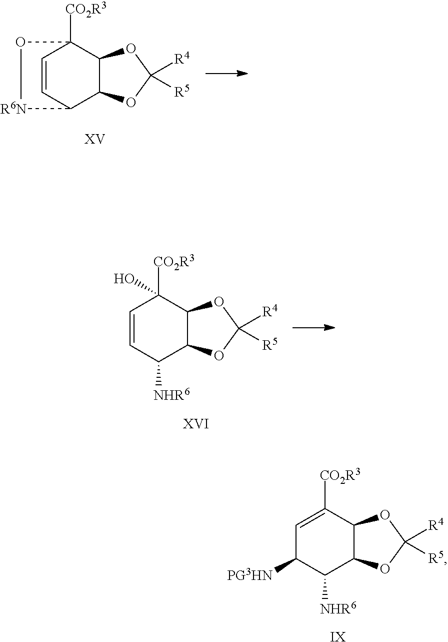

wherein, in the compounds of the formulae IX and XIII-XVI, one or more hydrogens in R³, R⁴, R⁵, and/or R⁶ is/are optionally replaced with F.

In a further alternate embodiment of the present application, there is included a process of preparing a compound of the formula XVIII

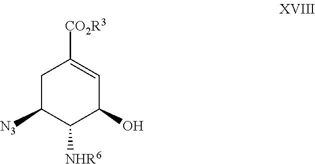

wherein R³ is C₁₋₆alkyl and R⁶ is C₁₋₆acyl, said process comprising:
(i) converting the hydroxyl group of a compound of the formula XVI, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, into a suitable leaving group in the presence of a base to provide a compound of formula XIX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

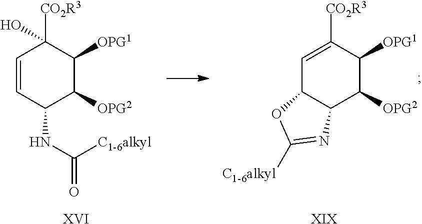

(ii) cleaving the oxazoline ring of the compound of formula XIX, followed by hydrogenation to provide a compound of the formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

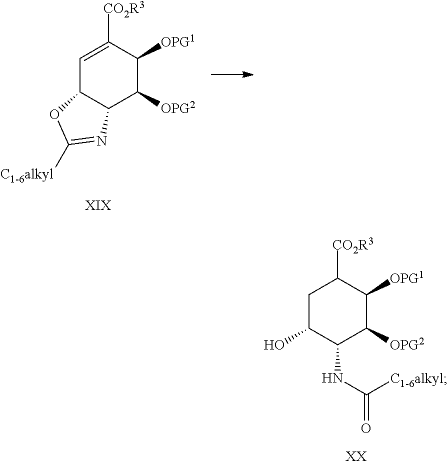

and (iii) converting the hydroxyl group of the compound of formula XX to a suitable leaving group, displacing the leaving group with azide and treating the resulting intermediate with a suitable base to provide a compound of the formula XVIII, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

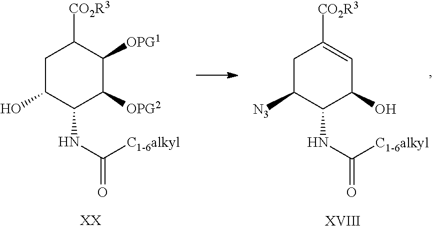

wherein, in the compounds of formulae XVI, XIX, XX and XVIII, one or more hydrogens in R³ and/or R⁶ is/are optionally replace by F.

The present application also includes a process for the preparation of compounds of formula XXI, wherein R³ is C₁₋₆alkyl and PG³ is a suitable protecting group

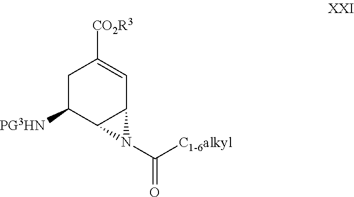

the process comprising:
(i) converting the hydroxyl group of a compound of formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, to a suitable leaving group, displacing the leaving group with a compound of the formula PG³NH₂ wherein PG³ is a suitable leaving group to provide a compound of the formula XXII, wherein R³ is C₁₋₆alkyl and PG¹, PG² and PG³ are suitable protecting groups and PG¹ and PG² are optionally linked to form, together with the oxygen atoms to which they are attached, an optionally C₁₋₆alkyl-substituted 5- or 6-membered ring,

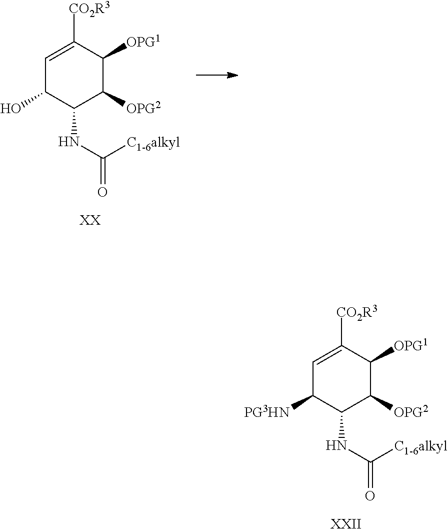

(ii) reducing double bond in the ring of the compound of formula XXII followed by treating the resulting saturated ring compound with a suitable base to provide a compound of the formula XI, wherein R³ is C₁₋₆alkyl and PG³ is a suitable protecting group,

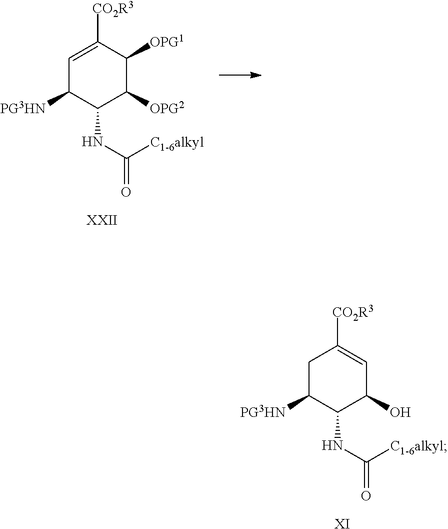

and
(iii) converting the hydroxyl group in the compound of formula XI to a leaving group and treating the resulting compound with a suitable base to provide a compound of the formula XXI, wherein R³ is C₁₋₆alkyl and PG³ is a suitable protecting group,

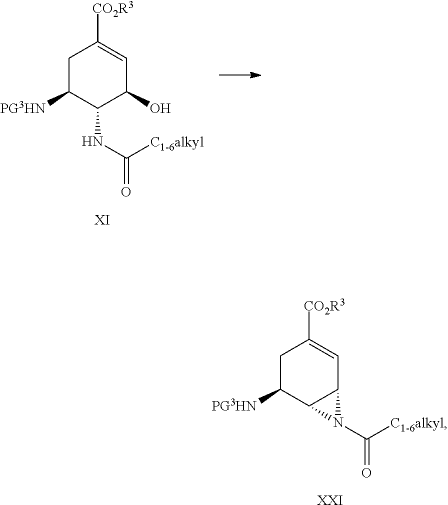

wherein in the compounds of the formulae XX, XXI, XXII and XI, R³ or C₁₋₆alkyl is/are optionally replaced with F.

Also within the scope of the present application are novel intermediate compounds for the preparation of oseltamivir and analogs thereof of the formula I. According to one aspect of the application, the novel intermediates are compounds of formula A:

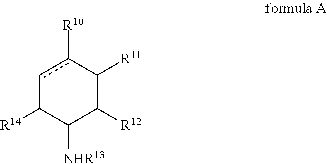

wherein,
R¹⁰ is selected from halo and CO₂R¹⁵;
R¹¹ is selected from OH and OR¹⁶;
R¹² is selected from OH, OR¹⁷ and N₃;
R¹³ is selected from H and C₁₋₆acyl;
R¹⁴ is selected from OC₁₋₆alkyl, SC₁₋₆alkyl, OH, SH, halo, N₃, NH₂, NHC₁₋₆alkyl and NHPG⁴, or
R¹³ and R¹⁴ are linked to form, together with the atoms to which they are attached, and oxazoline ring;
R¹⁵ is C₁₋₆alkyl;
R¹⁶ and R¹⁷ are the same or different and are, independently, PG⁵ or R¹⁶ and
R¹⁷ are joined together, to form, together with the oxygen atoms to which they are attached, a 5-membered cyclic ketal that is substituted on the carbon between the oxygen atoms by one or two C₁₋₆alkyl;
PG⁴ and PG⁵ are, independently protecting groups; [symbol]
represents a single or double bond, and
one or more hydrogens in the C₁₋₆alkyl and/or C₁₋₆acyl groups is/are is optionally replaced with F,
or salts, solvates, prodrugs, stereoisomers or isotope-labelled forms thereof,
or mixtures thereof,
provided that when R¹⁴ is NHPG⁴ or NHC₁₋₆alkyl, R¹⁰ is CO₂Et, R¹¹ is OH or OPG⁴ and PG⁴ is acyl, then R¹² is not 3-pentoxy.

In an embodiment of the application, the stereochemistry of the various chiral centers in the compounds of formula A is that required for the preparation of oseltamivir.

According to another aspect of the application, the novel intermediates for the preparation of oseltamivir and analogs thereof of the formula I are compounds of formula B:

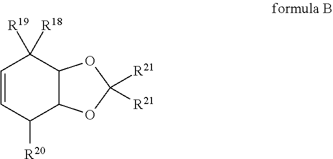

wherein,

### R¹⁸ is CO₂R²⁴;

R¹⁹ is selected from H, OH and OC₁₋₆acyl;
R²⁰ is NHC₁₋₆acyl; or the O in R¹⁹ and the N in R²⁰ are joined by a covalent bond;
R²¹ and R²² are, independently, C₁₋₆alkyl;
R²⁴ is C₁₋₆alkyl, and
one or more of the hydrogen atoms is the C₁₋₆alkyl and/or C₁₋₆acyl groups is/are optionally replaced with F,
or salts, solvates, prodrugs, stereoisomers or isotope-labelled forms thereof,
or mixtures thereof.

In an embodiment of the application, the stereochemistry of the various chiral centers in the compounds of formula B is that required for the preparation of oseltamivir.

In another aspect of the application, the compounds of formulae A and B are selected from:

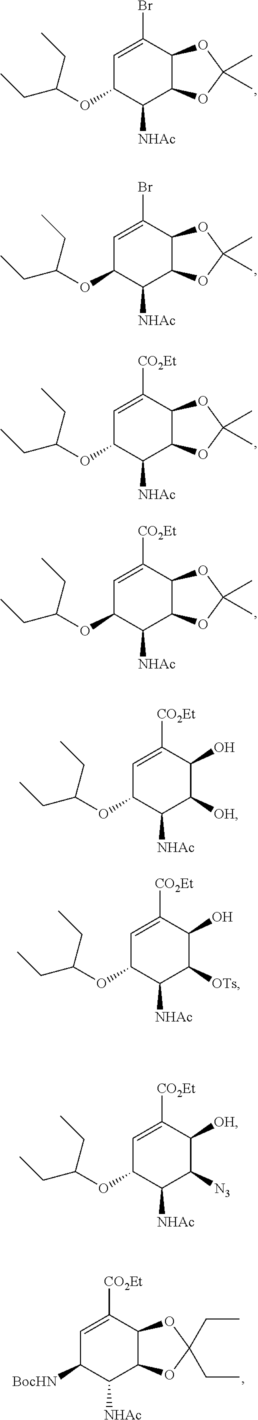

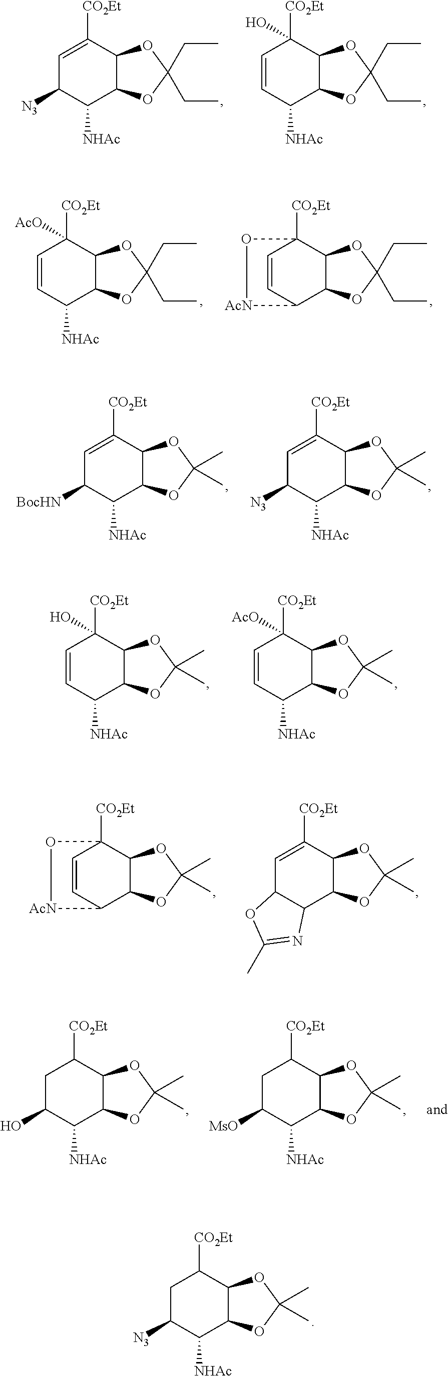

or salts, solvates, prodrugs, stereoisomers or isotope labeled forms thereof, or mixtures thereof.

According to another aspect of the application, there are provided pharmaceutical compositions comprising a compound of the application and a pharmaceutically acceptable carrier or diluent.

Another aspect of the application relates to a use of a compound of the application for the treatment or prevention of influenza.

Another aspect of the application relates to a use of a compound of the application for the preparation of a medicament for the treatment or prevention of influenza.

Also within the scope of the present application is a method of treating or preventing influenza comprising administering an effective amount of a compound of the application to a subject in need thereof.

The present application also includes a process for the preparation intermediates for the preparation of compounds of formula A. For example, the present application includes a process comprising:
(i) converting a compound of the formula XXIII, wherein R¹⁰ is selected from halo and CO₂R¹⁵ and R¹⁵ is C₁₋₆alkyl, to the diol of the formula XXIV, wherein R¹⁰ is selected from halo and CO₂R¹⁵ and R¹⁵ is C₁₋₆alkyl, by toluene dioxygenase mediated oxidation,

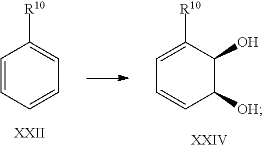

(ii) protecting the hydroxyl groups of the diol of formula XXIV, followed by aziridination to provide a compound of the formula XXV, wherein R¹⁰ is selected from halo and CO₂R¹⁵, R¹⁵ is C₁₋₆alkyl, R²³ is C₁₋₆acyl or PG⁵ and PG⁵, PG⁶ and PG⁷ are, independently, suitable protecting groups wherein PG⁶ and PG⁷ are optionally linked to form, together with the oxygen atoms to which they are attached, an optionally C₁₋₆alkyl-substituted 5- or 6-membered ring,

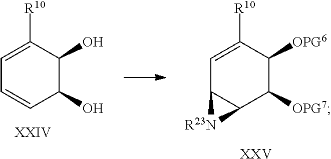

and
(iii) opening the aziridine ring in the compound of the formula XXV with a nucleophilic reagent that provides a source of ⁻OC₁₋₆alkyl, ⁻SC₁₋₆alkyl, ⁻OH, ⁻SH, ⁻halo, ⁻N₃, ⁻NH₂, ⁻NHC₁₋₆alkyl and NH-PG⁴, to provide a compound of the formula XXVI, wherein R¹⁰ is selected from halo and CO₂R¹⁵, R¹⁵ is C₁₋₆alkyl, R²³ is C₁₋₆acyl or PG⁵ and PG⁵, PG⁶ and PG⁷ are, independently, suitable protecting groups wherein PG⁶ and PG⁷ are optionally linked to form, together with the oxygen atoms to which they are attached, an optionally C₁₋₆alkyl-substituted 5- or 6-membered ring and R¹⁴ is OC₁₋₆alkyl, SC₁₋₆alkyl, OH, SH, halo, N₃, NH₂, NHC₁₋₆alkyl and NHPG⁴, and PG⁴ is a suitable protecting group,

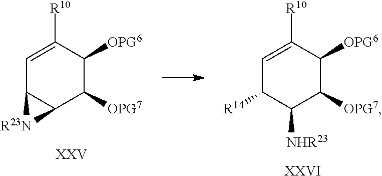

wherein, in the compounds of the formulae XXIII-XXVI, one or more hydrogens in C₁₋₆alkyl and/or C₁₋₆acyl is/are optionally replaced with F.

Compounds of the formula XXVI, represent a selection of the compounds of the formula A (i.e. wherein R¹⁰ is as defined in formula A, R¹¹ and R¹² are, independently, OPG⁵, R¹³ is C₁₋₆acyl, R¹⁴ is as defined in formula A and [symbol] represents double bond) and are converted to other compounds of formula A using methods known in the art.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

The application will now be described in greater detail with reference to the drawings in which:

FIG. 1 is a scheme showing a process for the preparation of oseltamvir according to an embodiment of the present application;

FIG. 2 is a scheme showing a process for the preparation of oseltamivir according to another embodiment of the present application;

FIG. 3 is a scheme showing a prophetic process for the preparation of oseltamivir according to another embodiment of the present application;

FIG. 4 is a scheme showing a process for the preparation of an intermediate to oseltamivir (a process for a partial synthesis of oseltamivir) according to an embodiment of the present disclosure;

FIG. 5 is a scheme showing further processes for the preparation of oseltamivir according to another embodiment of the present application; and

FIG. 6 is a scheme showing further processes for the preparation of intermediates for the preparation of oseltamivir according to another embodiment of the present application (broken arrows indicated prophetic reactions).

### DETAILED DESCRIPTION OF THE APPLICATION

### (I) Definitions

The term “C₁₋₆alkyl” as used herein means straight and/or branched chain, saturated alkyl groups containing 1, 2, 3, 4, 5, or 6 atoms and includes methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like. In the C₁₋₆alkyl groups, one or more, including all, of the hydrogen atoms may be replaced with F, and thus includes, for example trifluoromethyl, pentafluoroethyl and the like.

The term “C₁₋₆acyl” as used herein means straight or branched chain saturated acyl groups containing 1, 2, 3, 4, 5, or 6 carbon atoms. Examples of C₁₋₆acyl groups include but are not limited to CH₃CO, CH₃CH₂CO and the like. In the C₁₋₆acyl groups, one or more, including all, of the hydrogen atoms may be replaced with F, and thus includes, for example CF₃CO, CF₃CF₃CO, and the like

The term “halo” as used herein refers to a halogen atom and includes F, CI, Br and I.

The term “optionally C₁₋₆alkyl-substituted” as used herein means that the referenced group is either unsubstituted or substituted with one or more, suitably one or two, C₁₋₆alkyl groups.

The term “isotope-labelled forms” as used herein refers to compounds wherein one or more of the atoms has been substituted with an isotopic form of that atom that is other than the most abundant form of that atom in nature. For example a ¹²C atoms is replaced with a ¹⁴C or ¹³C atom, an ¹H atom is replace with a ²H or ³H atom or an ¹⁴N atom is replaced with ¹⁵N. In some cases, the isotope is a radioisotope.

In some cases the chemistries outlined herein may have to be modified, for instance by use of protecting groups, to prevent side reactions of reactive groups attached as substituents. This may be achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999.

The terms “protective group” or “protecting group” or “PG” or the like as used herein refer to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not destroy or decompose the molecule. Many conventional protecting groups are known in the art, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3^{rd} Edition, 1999. These may include but are not limited to Boc, Ts, Ms, TBDMS, TBDPS, Tf, Bn, allyl, Fmoc, C₁₋₆acyl, silyl, and the like.

The term “leaving group” as used herein refers to a group that is readily displaceable by a nucleophile, for example, under nucleophilic substitution reaction conditions. Examples of suitable leaving groups include, halo, Ms, Ts, Ns, Tf, Bn, C₁₋₆acyl, alkylsulphonyl and the like.

The term “suitable”, as in for example, “suitable protecting group”, “suitable leaving group” or “suitable reaction conditions” means that the selection of the particular group or conditions would depend on the specific synthetic manipulation to be performed and the identity of the molecule but the selection would be well within the skill of a person trained in the art. All process steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

Boc as used herein refers to the group t-butyloxycarbonyl.

Ac as used herein refers to the group acetyl.

Ts (tosyl) as used herein refers to the group p-toluenesulfonyl

Ms as used herein refers to the group methanesulfonyl

TBDMS as used herein refers to the group t-butyldimethylsilyl.

TBDPS as used herein refers to the group t-butyldiphenylsilyl.

Tf as used herein refers to the group trifluoromethanesulfonyl.

Ns as used herein refers to the group naphthalene sulphonyl.

Bn as used herein refers to the group benzyl.

In all of the compounds disclosed herein, that is compounds of the formulae I-XXVI, A and B, one or more, including all, of the hydrogen atoms is/are optionally replaced with F. A person skilled in the art would appreciate that only those hydrogens available for substitution by fluorine would be replaceable by fluorine.

The term “compound(s) of the application” or “intermediate compounds” used herein means compound(s) of formulae A and B as defined above, or any other novel intermediate compounds defined above, stereoisomers thereof or pharmaceutically acceptable salts, solvates or prodrugs thereof or isotope-labelled forms thereof, including mixtures thereof.

In embodiments of the application, the compounds of the application have at least one asymmetric centre. Where the compounds according to the application possess more than one asymmetric centre, they may exist as diastereomers. It is to be understood that all such isomers and mixtures thereof in any proportion are encompassed within the scope of the present application. It is to be understood that while the stereochemistry of the compounds of the application may be as provided for in any given compound listed herein, such compounds of the application may also contain certain amounts (e.g. less than 20%, suitably less than 10%, more suitably less than 5%) of compounds of the application having alternate stereochemistry.

The term “pharmaceutically acceptable” means compatible with the treatment of animals, in particular, humans.

The term “pharmaceutically acceptable salt” means an acid addition salt, which is suitable for, or compatible with, the treatment of patients.

The term “pharmaceutically acceptable acid addition salt” as used herein means any non-toxic organic or inorganic salt of any base compound of the application, or any of its intermediates. Illustrative inorganic acids, which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the application are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the application, for laboratory use, or for subsequent conversion to a pharmaceutically acceptable acid addition salt. In embodiments of the application, the pharmaceutically acceptable acid addition salt is the hydrochloride salt, or the H₃PO₄ salt. The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound of the application or a pharmaceutically acceptable salt of a compound of the application, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions.

Compounds of the application include prodrugs. In general, such prodrugs will be functional derivatives of a compound of the application which are readily convertible in vivo into the compound from which it is notionally derived. In an embodiment, prodrugs of the compounds of the application are conventional esters formed with available hydroxy, or amino groups. For example, an available OH or nitrogen in a compound of the application is acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the application are those in which one or more of the hydroxy groups in the compounds is masked as groups which can be converted to hydroxy groups in vivo. Conventional procedures for the selection and preparation of suitable prodrugs are described, for example, in “Design of Prodrugs” ed. H. Bundgaard, Elsevier, 1985.

Compounds of the application includes radiolabeled forms or isotope-labelled forms, for example, compounds of the application labeled by incorporation within the structure ³H, ¹³C or ¹⁴C or a radioactive halogen such as ¹²⁵I. A radiolabeled compound of the application may be prepared using standard methods known in the art. For example, tritium may be incorporated into a compound of the application using standard techniques, for example by hydrogenation of a suitable precursor to a compound of the application using tritium gas and a catalyst. Alternatively, a compound of the application containing radioactive iodo is prepared from the corresponding trialkyltin (suitably trimethyltin) derivative using standard iodination conditions, such as [¹²⁵I] sodium iodide in the presence of chloramine-T in a suitable solvent, such as dimethylformamide. In a further embodiment, the trialkyltin compound is prepared from the corresponding non-radioactive halo, suitably iodo, compound using standard palladium-catalyzed stannylation conditions, for example hexamethylditin in the presence of tetrakis(triphenylphosphine) palladium (0) in an inert solvent, such as dioxane, and at elevated temperatures, suitably 50-100° C.

To “inhibit” or “suppress” or “reduce” a function or activity, is to reduce the function or activity when compared to otherwise same conditions except for a condition or parameter of interest, or alternatively, as compared to another condition. The terms “inhibitor” and “inhibition”, in the context of the present application, are intended to have a broad meaning and encompass compounds of the application which directly or indirectly (e.g., via reactive intermediates, metabolites and the like) act on the influenza virus or symptoms thereof.

The term an “effective amount” or a “sufficient amount” of a compound as used herein is that amount sufficient to effect beneficial or desired results, including clinical results, and, as such, an “effective amount” depends upon the context in which it is being applied. For example, in the context of administering an agent that treats or prevents influenza, an effective amount of an agent is, for example, an amount sufficient to achieve a reduction in the amount of virus or of influenza symptoms as compared to the response obtained without administration of the agent.

It should be noted that the yields reported in Figures and in the experimental examples reported hereinbelow are non-limiting, unoptimized yields. A person skilled in the art would appreciate that reaction conditions will vary depending on a number of factors, including, for example, reaction scale and atmospheric conditions. The present application extends to processes that provide alternate yields (greater or less) of the desired products.

As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder.

The term “subject” as used herein includes all members of the animal kingdom including human. The subject is preferably a human.

### (II) Processes of the Application

The present application includes syntheses of oseltamivir, and analogs thereof, via a flexible symmetry-based design from the cis-dihydrodiol derived enzymatically from ethyl benzoate or bromobenzene. As shown in Scheme 2 below, oseltamivir contain a latent symmetry axis connecting C1 and C4 and this feature has been exploited herein, in a flexible design from to diasteromeric aziridines. In the representation shown in Scheme 2 the two structures are identical, however if the substituents at C3, C4 and C5 are not specifically defined then the configurations of these three carbons represent an enantiomeric switch that is controlled by the translocation of the double bond. This latent symmetry is exploited herein by designing an approach in which the order and the site of introduction of nitrogen and oxygen are interchanged. In approach A, the ether functionality is introduced to an activated allylic position, followed by inversion at C-5 with a nitrogen nucleophile and removal by reduction of the C-6 hydroxyl. In this approach the acrylate double bond remains in its original position. Conversely, it is the nitrogen functionality that is introduced in an activated allylic position in approach B, followed by a “symmetry switch” of the double bond with concomitant elimination of the C-6 hydroxyl and final alkylation of the C-5 hydroxyl. This latter approach has the advantage that the C-6 hydroxyl need not be removed by reduction but rather by elimination upon translocation of the olefin and creation of the allylic alcohol. Various strategies for activating the allylic position for reaction with either an oxygen or nitrogen functionality as shown in both approach A and B below have been used herein, including the formation of an aziridine ring (as shown) and formation of a double bond between the carbons 5 and 6 (available, for example, in short sequence via a hetero Diel's Alder reaction of the cis-dihydrodiol with an acyl nitroso compound, followed by reduction and further functionalization).

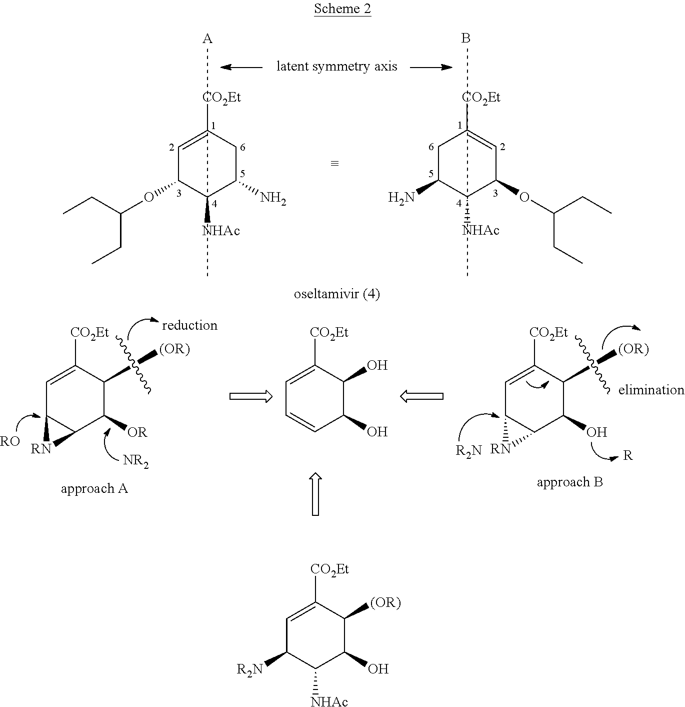

The present application therefore includes a first process for the preparation of oseltamivir and analogs thereof of the formula I:

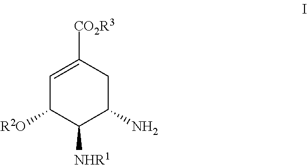

wherein R¹ is C₁₋₆acyl;
R² is C₁₋₆alkyl; and
R³ is C₁₋₆alkyl,
said process comprising:
(i) converting a halobenzene of the formula II, wherein X is halo, to the diol of the formula III, wherein X is halo, by toluene dioxygenase mediated oxidation,

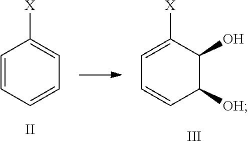

(ii) protecting the hydroxyl groups of the diol of formula III, followed by aziridination to provide a compound of the formula IV, wherein X is halo, R¹ is C₁₋₆acyl and PG¹ and PG² are suitable protecting groups that are optionally linked to form, together with the oxygen atoms to which they are attached, an optionally C₁₋₆alkyl-substituted 5- or 6-membered ring,

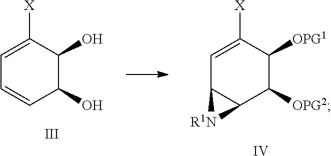

(iii) opening the aziridine ring in the compound of the formula IV with an alcohol of the formula R²—OH, wherein R² is C₁₋₆alkyl, followed by palladium catalyzed carbonylation to provide a compound of the formula V, wherein R¹ is C₁₋₆acyl, R² is C₁₋₆alkyl, R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

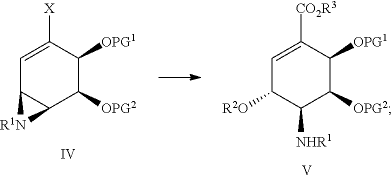

(iv) deprotecting the compound of the formula V and selectively converting the hydroxyl adjacent to the NHR¹ group to a leaving group (LG) to provide a compound of the formula VI, wherein R¹ is C₁₋₆acyl, R² is C₁₋₆alkyl, R³ is C₁₋₆alkyl and LG is a suitable leaving group,

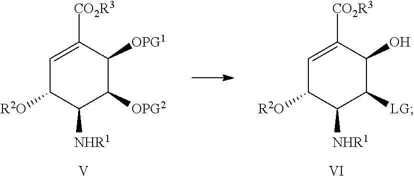

(v) displacing the leaving group in the compound of formula VI with an azide group to provide a compound of the formula VII, wherein R¹ is C₁₋₆acyl, R² is C₁₋₆alkyl and R³ is C₁₋₆alkyl,

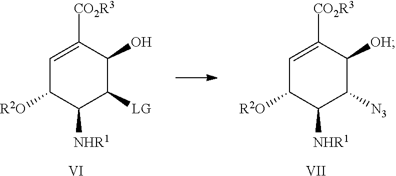

and
(vi) conversion of the hydroxyl group in the compound of the formula VII to a group removable by reduction and treating the resulting compound under reductive conditions to remove the group and converting the azide group to an amino group to provide a compound of formula I,

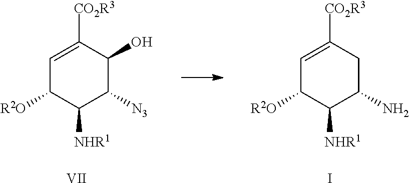

wherein, in the compounds of the formulae I-VII, one or more hydrogens in R¹, R² and/or R³ is/are optionally replaced with F.

In an embodiment of this first process, the compound of formula I is converted to its pharmaceutically acceptable salts. Conversion to the H₃PO₄ salt provides Tamiflu® and analogs thereof.

In an embodiment of the application, PG¹ and PG² in the compounds of formula IV and V are linked to form a cyclic ketal, suitably, the cyclic diethyl or dimethyl ketal more suitably the dimethyl ketal. In another embodiment X in the compounds of formulae II, III and IV is Br. In a further embodiment, R¹ is CH₃C(O), R² is (CH₃CH₂)₂CH and R³ is ethyl in the compounds of the formula IV, V, VI and VII.

In an embodiment of the application, the toluene dioxygenase mediated oxidation of step i of the first process is carried out in a whole-cell fermentation protocol using the recombinant strain E. coli JM109 (pDTG 601), for example, as described in Zylsra, G. J.; Gibson, D. T. J. Biol. Chem. 1989, 264, 14940.

In another embodiment of the first process, in step ii, the hydroxyl groups of the compound of the formula IV are protected as a cyclic ketal and the aziridation is carried out by reaction with N-bromoacetamide (NBA), tin bromide (SnBr₄) in a suitable solvent, for example acetonitrile, at a reduced temperature, for example about −20° C. to about −60° C., to form the intermediate III(a), followed by reaction with potassium hexamethyldisilazane (KHDMS) and tetrabutyl ammoniumbromide nBu₄NBr in a suitable solvent, for example, dimethoxyethane (DME) at a temperature of about −10° C. to about 10° C.

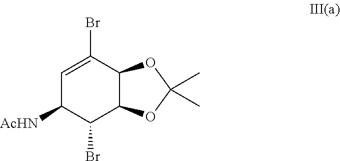

In a further embodiment of the first process, the aziridine is formed in step ii by reaction of the diene with an imino phenyliodinane reagent such as [N-(p-tolylsulfonyl)imino]phenyliodinane (PhI=NTs) or N-(acetylimino) phenyliodinane (PhI=NAc), with a catalyst, such as copper diacetylylacetonate (Cu(acac)₂), in a suitable solvent, such as acetonitrile.

In another embodiment of the first process, in step iii, the R²—OH ring opening of the compound of the formula IV is mediated by copper trifluoromethanesulfonate (Cu(OTf)₂).

In another embodiment of the first process, in step iii, the carbonylation of the compound of formula IV is performed in the presence of a metal catalysis, for example, Pd(PPh₃)₄ or Cl₂Pd(PPh₃)₂, in the presence of an organic base, such as Et₃N, in a suitable solvent, such as EtOH or PhMe, under about 1 atm of CO gas and at a temperature of about 40° C. to about 80° C.

In a further embodiment of the first process, in step iv, the leaving group, LG, is toluenesulfonyl, although a person skilled in the art that would appreciate that other leaving groups such as mesyl, would be equally suitable.

In a further embodiment of the first process, in step v, is performed by reacting the compound of formula VI with an azide salt, such as sodium azide in a suitable solvent, for example dimethylformide (DMF) under anhydrous conditions at a temperature of about 10° C. to about 40° C., suitably at about room temperature.

In another embodiment of the first process, in step vi, the hydroxyl group is reacted with methanesulfonyl chloride (MsCl) to form a mesyl group and the reduction is carried out using a hydride reducing agent such as from NaBH₄ or NaBH(OAc)₃ in a suitable solvent, such as ethanol, at a temperature of about −50° C. to about −10° C.

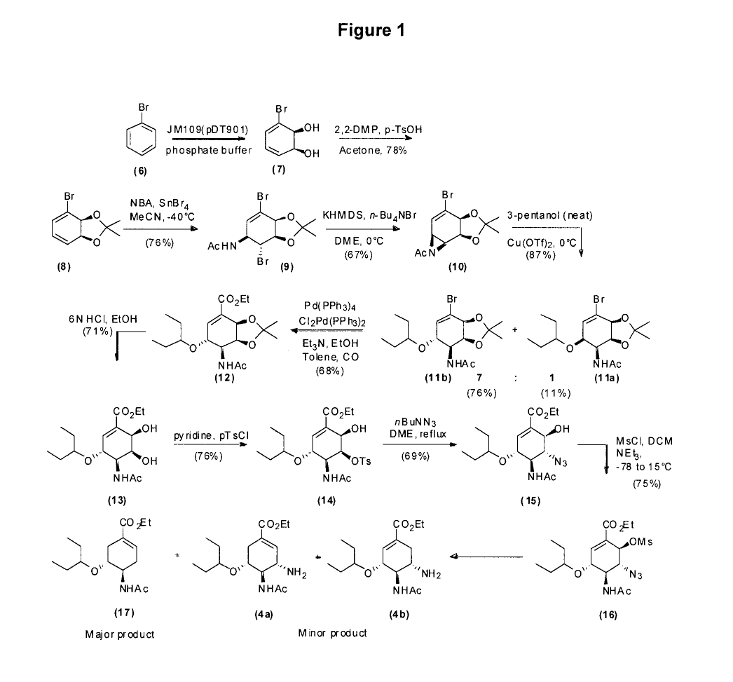

According to one specific embodiment of the application the first process is directed to the preparation of oseltamivir starting from bromobenzene as shown in FIG. 1. FIG. 1 shows only one embodiment of this process of the present application and the yields shown are non-limiting and un-optimized. Referring now to the compound numbers shown in FIG. 1, according to this embodiment, the starting material bromobenzene, 6, is converted to the diol 7 by toluene dioxygenase-mediate oxidation. In a particular embodiment the oxidation is performed using a whole cell fermentation protocol using the recombinant strain E. coli JM109(pDT601). In a further embodiment the oxidation with JM109(pDT601) is carried out in phosphate buffer. The asymmetry incorporated during the enzymatic step is transferred to the periphery of the diol 7 via an aziridination protocol, for example the protocol described in des Yeung, Y., Y; Hong, S. and Corey, E. J., J. Am. Chem. Soc. 2006, 128, 6310. In this protocol the diol is first protected to provide compound 8 and then converted to the aziridine in 2 steps—first by the formation of a brominated intermediate 9, for example by treatment with N-bromoacetamide (NBA) and SnBr₄, followed by ring closing displacement of the bromine to create the aziridine ring in 10 using and base, such as KHMDS and n-Bu₄NBr. In an embodiment, the aziridine is then ring opened with a C₁₋₆alkanol such as 3-pentanol, in the presence of a catalyst, such as coppertriflate, to provide the intermediates 11a and 11b. In a further embodiment, palladium catalyzed carbonylation of the enantiomer 11b provides the ethyl ester 12. In a further embodiment, deprotection of 12 is carried out with an acid, such as HCl, in a suitable solvent, such as ethanol, to give the diol 13. In another embodiment, the diol 13 is then selectively protected, for example with TsCl, to yield the intermediate 14, which is then, in another embodiment, displaced with sodium azide to give the azido alcohol 15. Finally, in yet another embodiment, mesylation and controlled low-temperature reduction provides as the major product, compound 17 with minor amounts of the regioisomers of oseltamivir, 4a and 4b. The reaction conditions for this latter reaction may be optimized to improve the yields of regioisomer 4a (oseltamivir). In an embodiment of the application, such optimized conditions include the use of chelating hydrides, such as B(OAc)₃HNa to comprise the reductive conditions in step vi of the first process of the application and/or converting the azide group to the amino group prior to treatment under reductive conditions.

In another aspect of the application there is provided a second process for the preparation of oseltamivir and analogs thereof of the formula I:

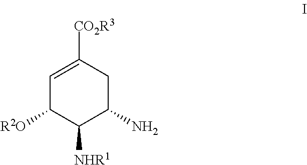

wherein R¹ is C₁₋₆acyl;
R² is C₁₋₆alkyl; and
R³ is C₁₋₆alkyl,
said process comprising:
(i) protecting the hydroxyl groups of a compound of formula III, wherein X is halo, as the di(C₁₋₆)alkyl ketal followed by aziridination to provide a compound of the formula VIII, wherein X is halo, R⁴ and R⁵ are independently, C₁₋₆alkyl and R⁶ is C₁₋₆acyl,

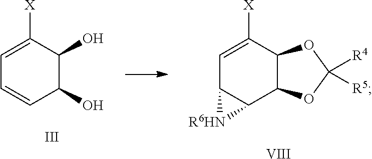

(ii) ring opening the aziridine ring of the compound of formula VIII with PG³-NH₂, where PG³ is a suitable protecting group, followed by palladium catalyzed carbonylation to provide a compound of the formula IX, wherein R³, R⁴ and R⁵ are, independently, C₁₋₆alkyl, R⁶ is C₁₋₆acyl and PG³ is a suitable protecting group,

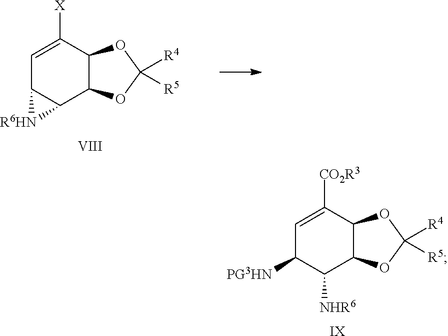

(iii) regioselective ring-opening of the cyclic ketal in the compound of formula IX and reduction to provide a compound of the formula X, wherein R³, R⁴ and R⁵ are, independently, C₁₋₆alkyl, R⁶ is C₁₋₆acyl and PG³ is a suitable protecting group,

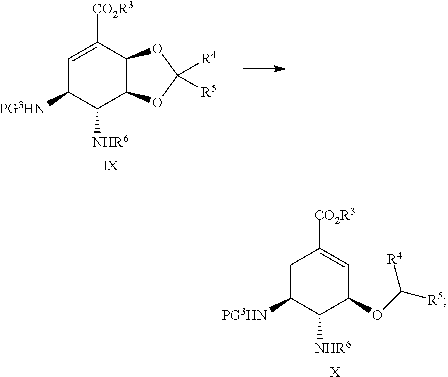

or, removing the cyclic ketal in the compound of formula IX followed by rearranging and eliminating one hydroxy group to provide a compound of the formula XI, wherein R³ is C₁₋₆alkyl, R⁶ is C₁₋₆acyl and PG³ is a suitable protecting group followed by alkylating the remaining hydroxy group with a compound of the formula R²-LG, wherein R² is C₁₋₆alkyl and LG is a suitable leaving group, to provide a compound of the formula XII, wherein R³ is C₁₋₆alkyl, R⁶ is C₁₋₆acyl and PG³ is a suitable protecting group,

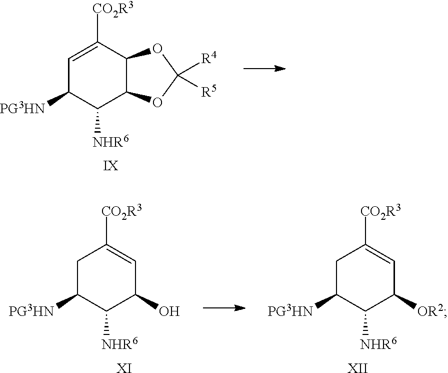

(iv) removing the PG³ of the compound of the formula X or XII to provide a compound of the formula I

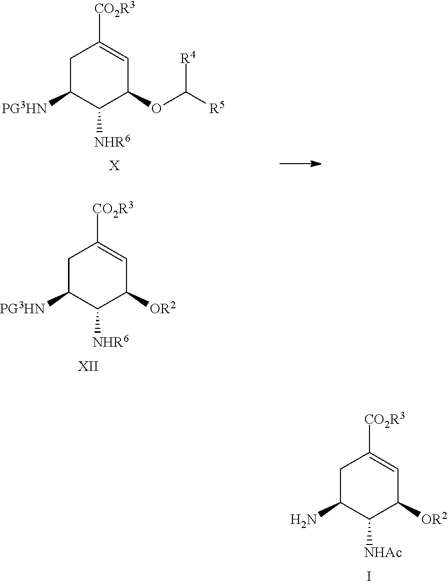

wherein, in the compounds of the formulae I, III and VIII-XII, one or more hydrogens in R¹, R², R³, R⁴, R⁵, and/or R⁶ is/are optionally replaced with F.

In another embodiment of this second process, the compound of the formula I is converted to its pharmaceutically acceptable salts. Conversion to the H₃PO₄ salt provides Tamiflu® and analogs thereof.

In embodiment of the application X in the compounds of formulae III and VII is Br. In another embodiment of the application, R³ in the compounds of the formulae I, IX, X, XI and XII is ethyl. In a further embodiment, R⁴ and R⁵ in the compounds of formulae VIII, IX and X are both ethyl. In a further embodiment, R⁶ in the compounds of the formulae I, VIII, IX, X, XI and XII is Ac. In yet another embodiment of the application PG³ in the compounds of the formulae IX, X, XI and XII is t-butoxycarbonyl (BOC).

According to another embodiment of the second process, in step i, the diol is protected as the diethyl cyclic ketal, and the aziridine is formed by reaction of the diene with an imino phenyliodinane reagent such as [N-(p-tolylsulfonyl)imino]phenyliodinane (PhI=NTs) or N-(acetylimino) phenyliodinane (PhI=NAc), with a catalyst, such as copper diacetylylacetonate (Cu(acac)₂), in a suitable solvent, such as acetonitrile. In an alternate embodiment, the aziridine of formula VIII is preparation by reaction with N-bromoacetamide (NBA), tin bromide (SnBr₄) followed by reacting with potassium hexamethyldisilazane (KHDMS) and tetrabutyl ammoniumbromide nBu₄NBr as described above for the preparation of the compound of formula IV.

According to another embodiment of the second process, in step ii, the aziridine opening with PG³-NH₂ is catalyzed by Cu(OTf)₂ and the carbonylation is catalyzed by a metal catalysis, such as Pd(PPh₃)₄, Cl₂Pd(PPh₃)₂, in the presence of an organic base, such as Et₃N, in a suitable solvent, such as EtOH, and PhMe under about 1 atm of CO gas at a temperature of about 40° C. to about 80° C.

In another embodiment of the second process, in step iii, the regioselective ring opening and reduction conditions comprise the use of Stryker's reagent [(Ph₃P)CuH]₆ carried out in the presence of triethylsilane (Et₃SiH) in a suitable solvent under conditions to provide a compound of formula X.

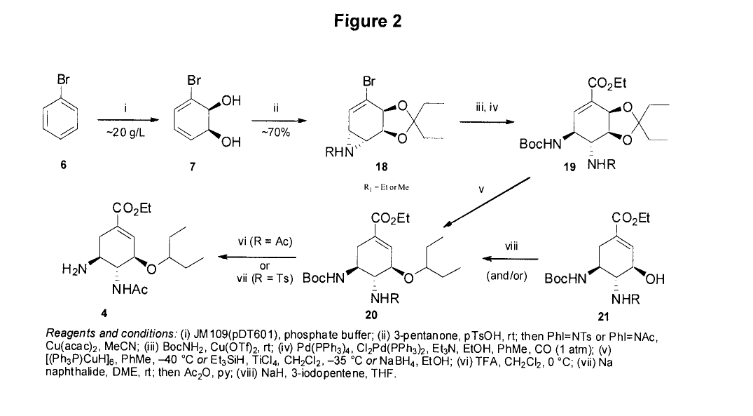

According to another specific embodiment of the application the second process is directed to the preparation of oseltamivir starting from bromobenzene as shown in FIG. 2. Referring now to the compound numbers shown in FIG. 2, in this embodiment, dihydroxylation of bromobenzene is carried out as in the first process as shown in FIG. 1. The diol 7 is protected, for example as a diethyketal (to form 2,2-diethyl-1,3-dioxolone). In this embodiment the aziridine is formed anti to the diol using, for example, either PhI=NTs or PhI=NAc and Cu(acac)₂ to give the intermediate 18 by the Yamada-Jacobsen-Evans protocol, (Yamada, Y. et al., Chem. Let. 1975, 361; Evans et al., J. Org. Chem. 1991, 56, 6744; Li et al. J. Am. Chem. Soc. 1993, 115, 5326; Li et al. J. Am. Chem. Soc. 1995, 117, 5889). Opening of the aziridine with, for example, BocNH₂ provides the intermediate 19. 1,4-Reduction of the acrylate with Stryker's reagent generates an intermediate anion that ejects the proximal C—O bond of the 1,3-dioxolone as shown in FIG. 2. Carried out in the presence of Et₃SiH this reaction is expected to give the correct topology for oseltamivir, as shown in the penultimate compound 20. Alternatively, intermediate 19 is converted to the allylic alcohol 21 and then alkylated to obtain intermediate 20. Deprotection with, for example, TFA to remove the Boc group provides, oseltamivir 4. Which is converted to the H₃PO₄ salt form, Tamiflu®.

In another aspect, the present application also includes an alternate process for the preparation of compounds of formula IX (used in the second process for the preparation of oseltamivir and analogs thereof of the formula I)

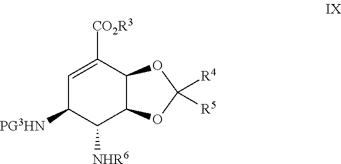

wherein R³, R⁴ and R⁵ are, independently, C₁₋₆alkyl, R⁶ is C₁₋₆acyl and PG³ is a suitable protecting group, said process comprising:
(i) converting a benzoate ester of formula XIII, wherein R³ is C₁₋₆alkyl, to a compound of the formula XIV, wherein R³ is C₁₋₆alkyl, by toluene dioxygenase mediated oxidation,

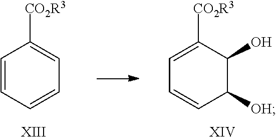

(ii) protecting the hydroxyl groups of the compound of formula XIV as the di(C₁₋₆alkyl) ketal followed by regio- and stereoselective nitroso Diels-Alder cycloaddition to the conjugated diene to provide a compound of the formula XV, wherein R³, R⁴ and R⁵ are, independently, C₁₋₆alkyl and R⁶ is C₁₋₆acyl,

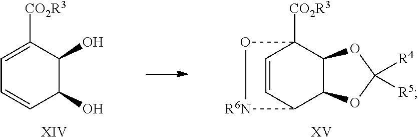

and
(iii) displacing the oxazine bridge of the compound of formula XV with PG³NH₂, wherein PG³ is a suitable protecting group, under palladium catalyzed conditions followed by reduction to provide a compound of the formula IX, wherein R³, R⁴ and R⁵ are, independently, C₁₋₆alkyl and R⁶ is C₁₋₆acyl and PG³ is a suitable protecting group,

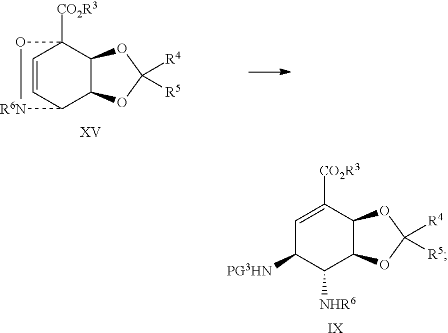

or,
ring opening of the oxazine bridge of the compound of formula XV to yield the compound of formula XVI, followed by converting the hydroxy group in the compound of formula XVI to a leaving group and allylic displacement of the leaving group with PG³NH₂ or azide, wherein PG³ is a suitable protecting group, to provide, after reduction of the azide and protection of the resulting amine with PG³, a compound of the formula IX, wherein R³, R⁴ and R⁵ are, independently, C₁₋₆alkyl and R⁶ is C₁₋₆acyl and PG³ is a suitable protecting group,

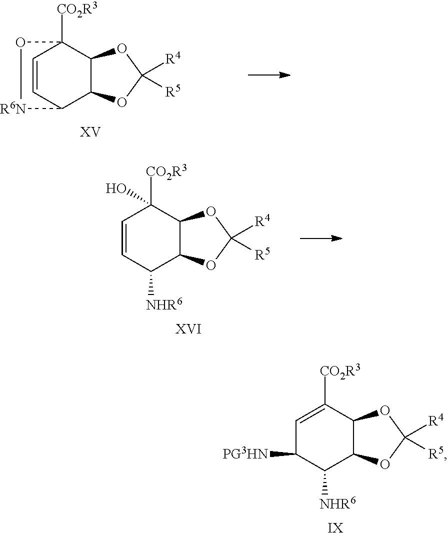

wherein, in the compounds of the formulae IX and XIII-XVI, one or more hydrogens in R³, R⁴, R⁵ and/or R⁶ is/are optionally replaced with F.

In an embodiment of the application R³ in the compounds of formulae IX, XIII, XIV, XV and XVI is methyl or ethyl, suitably methyl. In a further embodiment R⁴ and R⁵ in the compounds of formula IX, XV and XVI is methyl. In another embodiment, R⁶ in the compounds of formulae IX, XV and XVI is methyl. In another embodiment PG³ in the compounds of formula IX is t-butoxycarbonyl (t-Boc)

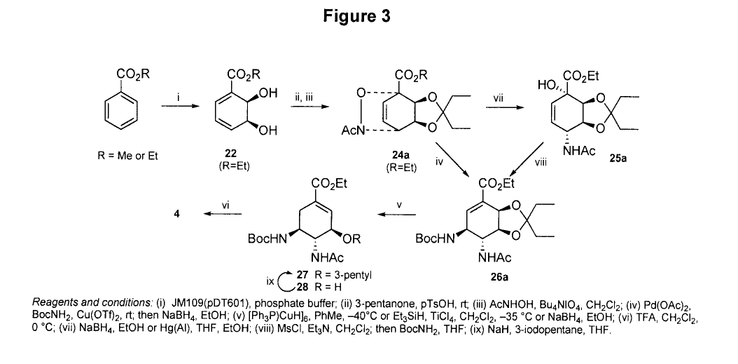

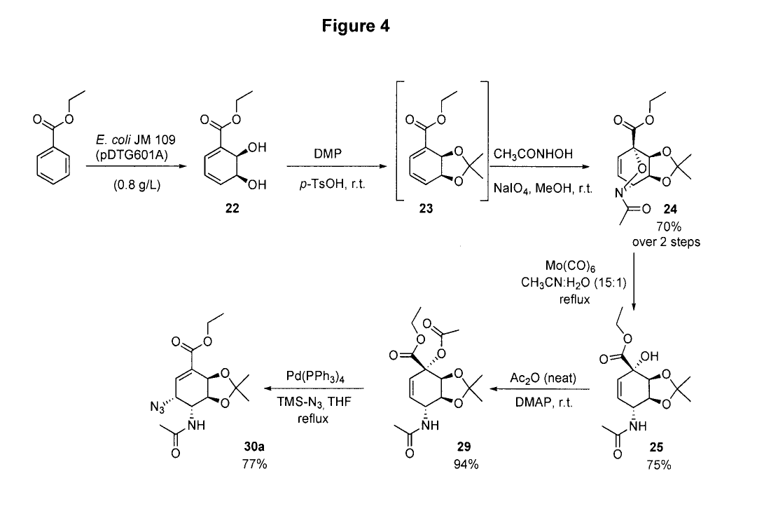

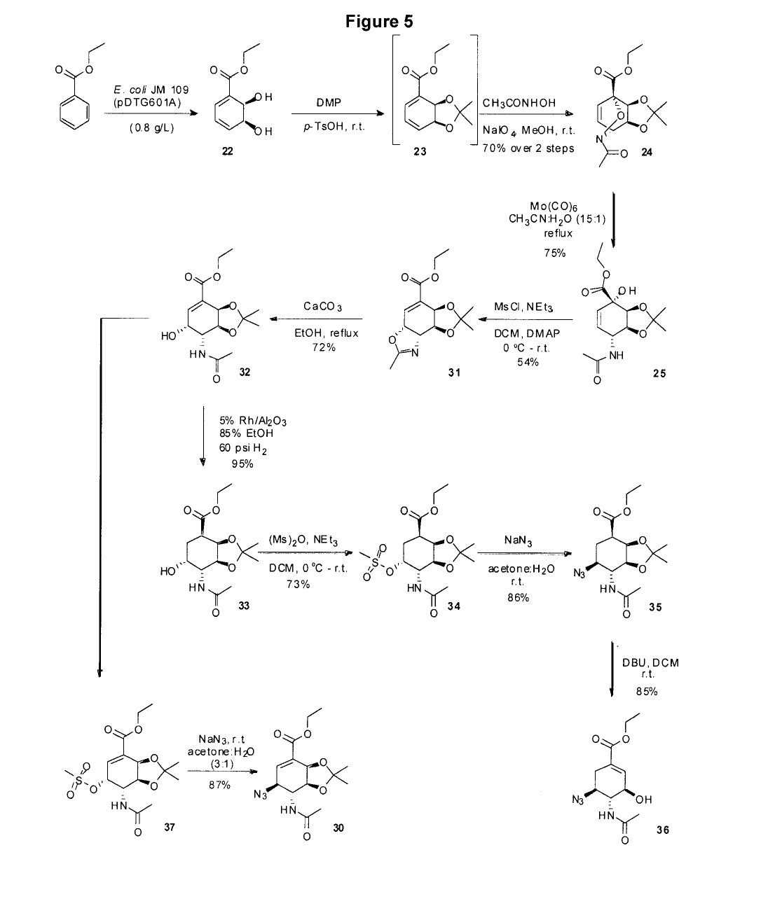

According to another specific embodiment of the application the alternative process for the preparation of compounds of the formula IX is shown in FIGS. 3 and 4 and partially in FIG. 5. FIGS. 3, 4 and 5 show only one embodiment of these processes of the present application and the yields shown are non-limiting and un-optimized. Referring now to the compound numbers in FIGS. 3, 4 and 5, this process starts with a benzoate ester, such as, but not limited to ethyl benzoate or methyl benzoate. Any benzoate ester may be used in this process, as the ester may be converted to the desired ethyl ester using a standard transesterification reaction as described in Fabris, F. et al. Org. Biomol. Chem. 2009, DOI: 10,1039b902577b. In a particular embodiment the benzoate ester is the ethyl benzoate. In this embodiment, the benzoate ester is converted to the diol, for example 22, by toluene dioxygenase-mediate oxidation. In a particular embodiment the oxidation is in a whole cell fermentation protocol using the recombinant strain E. coli JM109(pDT601). In a further embodiment the oxidation with JM109(pDT601) is carried out in phosphate buffer. The diol is then protected, for example, as a cyclic ketal using known procedures. In one embodiment the diol is protected with 3-pentanone in the presence of an acid to give the diethyl ketal (FIG. 3). In another embodiment the diol is protected with 2,2-dimethoxypropane (DMP) in the presence of an acid to give the dimethyl ketal (FIGS. 4 and 5). In a further embodiment, the resulting conjugated diene is subjected to a regio- and stereoselective hetero Diels-Alder cycloaddition to provide, for example, the intermediate 24a (FIG. 3) or 24 (FIGS. 4 and 5). In an embodiment, the hetero Diels Alder reaction is carried out by reacting the diene of a compound of Formula XIV, for example compound 23, with an acyl nitroso compound, such as N-hydroxyacetamide, in the presence of sodium periodate in a suitable solvent, such as ethanol, at a temperature in the range of about 10° C. to about 30° C., suitably about room temperature. In FIG. 3, the Diels-Alder cycloaddition product 24a is reacted directly with BocNH₂ in the presence of Pd(OAc)₂ and Cu(OTf)₂, then reduced with NaBH₄ to give the Boc protected amine 26a. In an alternative embodiment, this transition is carried out in two steps: first ring-opening with NaBH₄ or Hg (Al) to give the alcohol 25a, followed by conversion of the alcohol to a leaving group, in this case mesyl, followed by displacement with a nitrogen-containing nucleophile, in this case BocNH₂, to give intermediate 26a. In an embodiment, conversion to oseltamivir is affected as described for the second process or as shown in FIG. 3 where regio-selective ring-opening of the cyclic ketal with [(Ph₃P)CuH]₆ in toluene or Et₃SiH, TiCl₄ and reduction with NaBH₄ provides the intermediate 27 or the intermediate 28. If the intermediate 27 is obtained, alkylation of the alcohol provides 27, which is converted to oseltamivir by removal of the Boc protecting group using known procedures. In a further alternate embodiment, as shown in FIGS. 4 and 5, the Diels-Alder cycloaddition product 24 is reductively ring opened by reaction with, for example Mo(CO)₆, in a suitable solvent, for example acetonitrile/water at an elevated temperature, such as under reflux, to provide intermediate 25. In a further embodiment, conversion of the hydroxy group to a leaving group, such as acetyl, and reaction with a nucleophile, in the case of FIG. 4 and azide, in the presence of provides intermediate 30, which is reduced and protected to provide the intermediate of the formula IX.

In a further alternate embodiment of the present application, there is included a process of preparing a compound of the formula XVIII

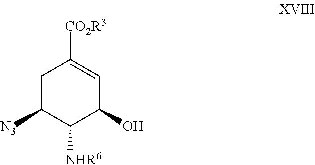

wherein R³ is C₁₋₆alkyl and R⁶ is C₁₋₆acyl, said process comprising:
(i) converting the hydroxyl group of a compound of the formula XVI, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, into a suitable leaving group in the presence of a base to provide a compound of formula XIX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

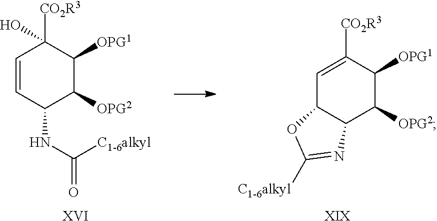

(ii) cleaving the oxazoline ring of the compound of formula XIX, followed by hydrogenation to provide a compound of the formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring,

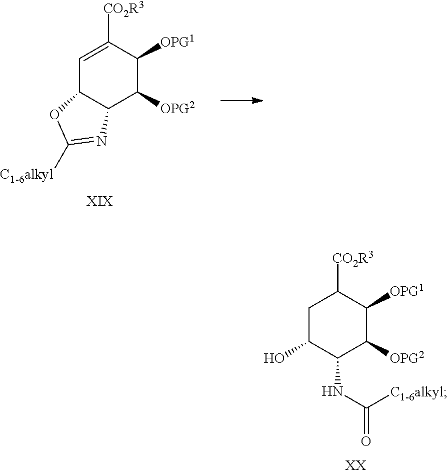

and
(iii) converting the hydroxyl group of the compound of formula XX to a suitable leaving group, displacing the leaving group with azide and treating the resulting intermediate with a suitable base to provide a compound of the formula XVIII, wherein R³ is C₁₋ₑalkyl and PG¹ and PG² are suitable protecting groups that are optionally linked to form, together with the oxygen atoms to which they are attached, an optionally C₁₋₆alkyl-substituted 5- or 6-membered ring,

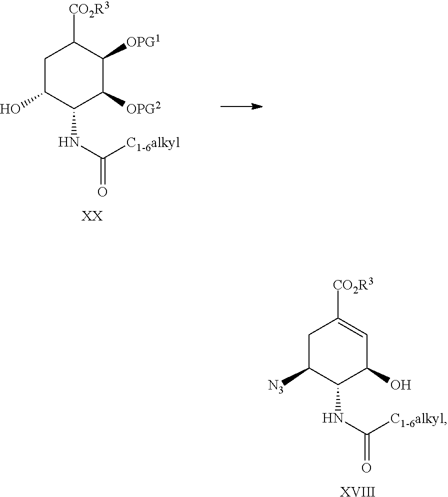

wherein, in the compounds of formulae XVI, XIX, XX and XVIII, one or more hydrogens in R³ and/or R⁶ is/are optionally replaced with F

In an embodiment of the application, R³ in the compounds of the formulae XVIII, XVI, XIX and XX is methyl or ethyl, suitably ethyl. In a further embodiment, R⁶ or C₁₋₆ alkyl in the compounds of formulae XVIII, XVI, XIX and XX is methyl. In another embodiment, PG¹ and PG² in the compounds of formulae XVI, XIX and XX are linked to form, together with the oxygen to which they are attached a dimethyl acetonide.

In an embodiment of the above process for the preparation of compounds of formula XVIII, the compound of formula XVI is obtained using the above-described process. However, in this embodiment, the compound of formula XVI is treated with mesityl chloride or another suitable leaving group reagent, in the presence of a base, such as an organic amine, and a catalytic amount of dimethylaminopyridine (or its equivalent) in a suitable organic solvent at a temperature of about 10° C. to about 30° C., suitably at about room temperature, which provides an oxazoline of the formula XIX, for example compound 31 in FIG. 5.

In a further embodiment of the above process for the preparation of compounds of formula XVIII, step ii is performed by treating the compound of the formula XIX with an inorganic base, such as calcium carbonate in a polar organic solvent, such as an alcohol, at elevated temperatures, for example at refluxing temperature. In a further embodiment, hydrogenation in step ii is performed using standard hydrogenation conditions, to provide the compounds of the formula XX.

In a further embodiment of the above process for the preparation of compounds of formula XVIII, step iii is performed by converting the hydroxy group in the compounds of the formula XX to a suitable leaving group, such as mesityl, using known conditions and displacement of the leaving group with azide, for example by treating with an azide salt, such as sodium azide, in a suitable solvent at a temperature of about 10° C. to about 30° C., suitably at about room temperature, followed by treatment with suitable base, such as an organic amine base, in a suitable solvent at a temperature of about 10° C. to about 30° C., suitably at about room temperature, provides compounds of the formula XVIII.

The compounds of the formula XVIII include Fang's intermediate which has previously been converted to oseltamivir Shie, J.-J.; Fang, J.-M.; Wong, C.-H. Angew. Chem. Int. Ed. 2008, 47:5788. The above process for the preparation of compounds of formula XVIII, includes within its scope, a formal synthesis of oseltamivir in seven operations from ethyl benzoate.

The present application also includes an “azide free” process for the preparation of compounds of formula XXI, wherein R³ is C₁₋₆alkyl and PG³ is a suitable protecting group

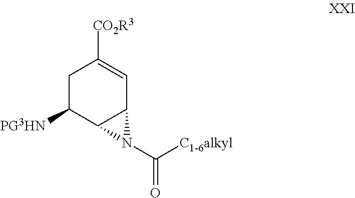

the process comprising:
(i) converting the hydroxyl group of a compound of formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, to a suitable leaving group, displacing the leaving group with a compound of the formula PG³NH₂ wherein PG³ is a suitable leaving group to provide a compound of the formula XXII, wherein R³ is C₁₋₆alkyl and PG¹, PG² and PG³ are suitable protecting groups and PG¹ and PG² are optionally linked to form, together with the oxygen atoms to which they are attached, an optionally C₁₋₆alkyl-substituted 5- or 6-membered ring,

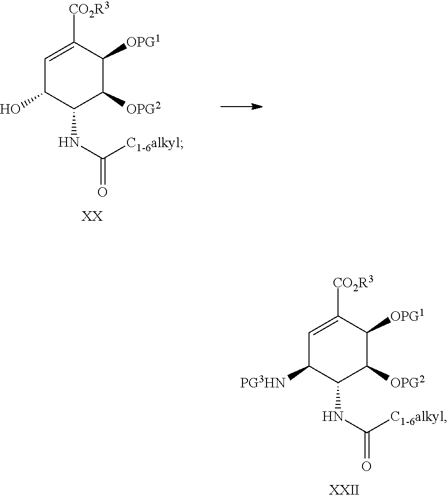

(iii) reducing double bond in the ring of the compound of formula XXII followed by treating the resulting saturated ring compound with a suitable base to provide a compound of the formula XI, wherein R³ is C₁₋₆alkyl and PG³ is a suitable protecting group,

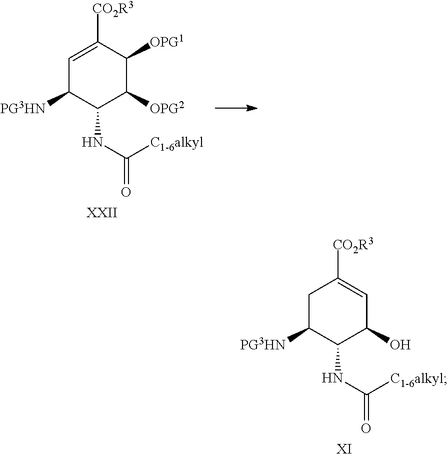

and
(iii) converting the hydroxyl group in the compound of formula XI to a leaving group and treating the resulting compound with a suitable base to provide a compound of the formula XXI, wherein R³ is C₁₋₆alkyl and PG³ is a suitable protecting group

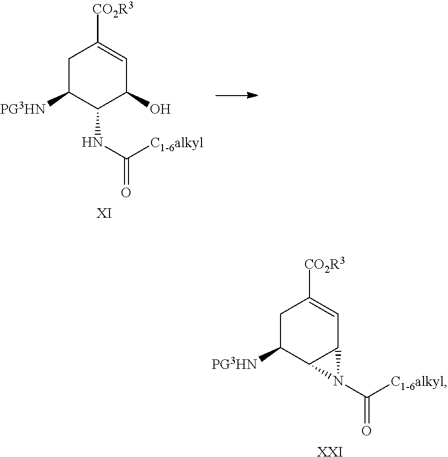

wherein in the compounds of the formulae XX, XXI, XXII and XI, one or more hydrogen is/are optionally replaced with F.

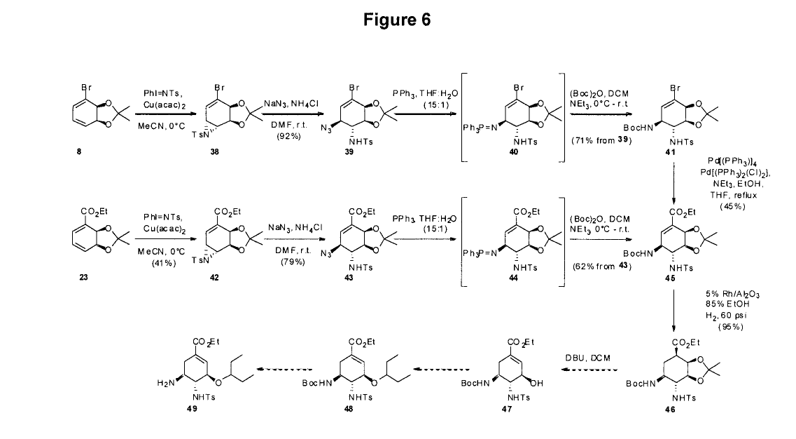

In an embodiment of the present application, R³ in the compounds of formulae XXI, XX, XXII and XI is methyl or ethyl, suitably ethyl. In a further embodiment, PG³ in the compounds of formulae XXI, XXII and XI is t-butoxycarbonyl (t-BOC). In another embodiment, PG¹ and PG² in the compounds of formulae and XX, XXII are linked to form, together with the oxygen atoms to which they are attached, a dimethyl acetonide. In an alternative embodiment of this “azide free” process to prepare the compounds of the formula XXI, the compound of the formula XX is converted to a suitable leaving group and is reacted with phthalimide or ammonia (NH₃). Reaction with phthalimide provides a phthalimido protected version of the compounds of formula XXII (i.e. where the PG³NH group is replaced with a phthalimido group). Reaction with ammonia, provides the corresponding amino compound that is suitably protected prior to further chemical manipulations. It is a further embodiment of this “azide free” process that, as shown in FIG. 6, the double bond in the compounds of the formulae XXII is reduced using standard hydrogenation conditions (e.g. H₂ in the presence of a transition metal catalyst). In a further embodiment, treatment of the resulting saturated compound in the presence of an organic amine base, for example, diaza(1,3)bicyclo[5.4.0]undecane, results formation of the double bond and elimination of one hydroxyl group to provide the compounds of the formula XI (e.g. compound 47 in FIG. 6). In another embodiment, the aziridine compounds of the formula XXI are generated under Mitsunobu conditions which can be converted to oseltamivir, and analogs thereof, by nucleophilic opening with a C₁₋₆alkanol, such as 3-pentanol.

### (III) Compounds of the Application

Also within the scope of the present application are novel intermediate compounds for the preparation of oseltamivir and analogs thereof of the formula I. According to one aspect of the application, the novel intermediates are compounds of formula A:

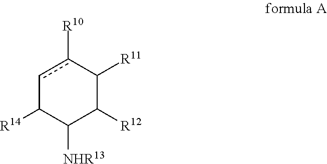

R¹⁰ is selected from halo and CO₂R¹⁵;
R¹¹ is selected from OH and OR¹⁶;
R¹² is selected from OH, OR¹⁷ and N₃;
R¹³ is selected from H and C₁₋₆acyl;
R¹⁴ is selected from OC₁₋₆alkyl, SC₁₋₆alkyl, OH, SH, halo, N₃, NH₂, NHC₁₋₆alkyl and NHPG⁴, or
R¹³ and R¹⁴ are linked to form, together with the atoms to which they are attached, and oxazoline ring;
R¹⁵ is C₁₋₆alkyl;
R¹⁶ and R¹⁷ are the same or different and are, independently, PG⁵ or R¹⁶ and
R¹⁷ are joined together, to form, together with the oxygen atoms to which they are attached, a 5-membered cyclic ketal that is substituted on the carbon between the oxygen atoms by one or two C₁₋₆alkyl;
PG⁴ and PG⁵ are, independently protecting groups; [symbol]
represents a single or double bond, and
one or more hydrogens in the C₁₋₆alkyl and/or C₁₋₆acyl groups is/are optionally replaced with F,
or salts, solvates, prodrugs, stereoisomers or isotope-labelled forms thereof,
or mixtures thereof,
provided that when R¹⁴ is NHPG⁴ or NHC₁₋₆alkyl, R¹⁰ is CO₂Et, R¹¹ is OH or OPG⁴ and PG⁴ is acyl, then R¹² is not 3-pentoxy.

In an embodiment of the application, the stereochemistry of the various chiral centers in the compounds of formula A is that required for the preparation of oseltamivir.

In a further embodiment of the application, R¹⁰ in the compounds of formula A is Br. In other embodiments of the application R¹⁰ in the compounds of formula A is CO₂Me or CO₂Et, suitably CO₂Et.

In another embodiment of the present application, R¹¹ and R¹² in the compounds of formula A are both OH. In a further embodiment, R¹¹ and R¹² in the compounds of formula A are OR¹⁶ and OR¹⁷, respectively, where R¹⁶ and R¹⁷ are joined together, to form, together with the oxygen atoms to which they are attached, a 5-membered dimethyl or diethyl ketal, suitably a dimethyl ketal.

In another embodiment of the present application, R¹³ in the compounds of formula A is H or C(O)CH₃, suitably H.

In another embodiment of the present application, R¹⁴ in the compounds of formula A is selected from OH, NH₂, OC₁₋₆alkyl, SC₁₋₆alkyl and NHC₁₋₆alkyl.

In another embodiment of the present application [symbol] in the compounds of formula A represents a double bond. In a further embodiment, [symbol] represents a single bond.

In another embodiment of the present application R¹³ and R¹⁴ in the compounds of formula A are linked to form, together with the atoms to which they are attached, and oxazoline ring.

According to another aspect of the application, the novel intermediates for the preparation of oseltamivir and analogs thereof of the formula I are compounds of formula B:

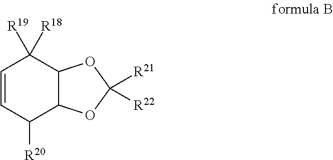

wherein,

### R¹⁸ is CO₂R²⁴;

R¹⁹ is selected from H, OH and OC₁₋₆acyl;
R²⁰ is NHC₁₋₆acyl;
or the 0 in R¹⁹ and the N in R²⁰ are joined by a covalent bond;
R²¹ and R²² are, independently, C₁₋₆alkyl;
R²⁴ is C₁₋₆alkyl; and
one or more of the hydrogens in the C₁₋₆alkyl and/or C₁₋₆acyl groups is/or optionally replaced with F,
or salts, solvates, prodrugs, stereoisomers or isotope-labelled forms thereof, or mixtures thereof.

In an embodiment of the application, the stereochemistry of the various chiral centers in the compounds of formula B is that required for the preparation of oseltamivir.

In another embodiment of the application R¹⁸ in the compounds of formula B is CO₂Et or CO₂Me, suitably CO₂Et.

In yet another embodiment, R¹⁹ in the compounds of formula B is OH or OC(O)CH₃.

In another embodiment of the present application, R²⁰ in the compounds of formula B is NHC(O)CH₃.

In another embodiment, the 0 in R¹⁹ and the N in R²⁰ in the compounds of formula B are joined by a covalent bond to form a bridged bicyclic compound.

In another embodiment of the present application, R²¹ and R²² in the compounds of formula B are both methyl or ethyl.

In another aspect of the application, the compounds of formula A and B are selected from:

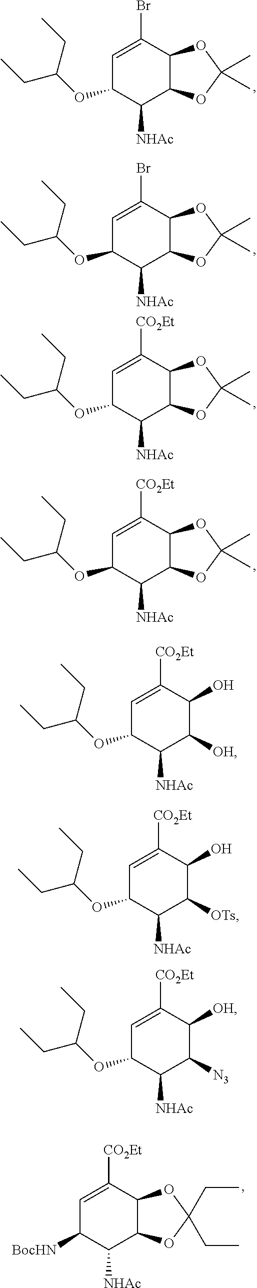

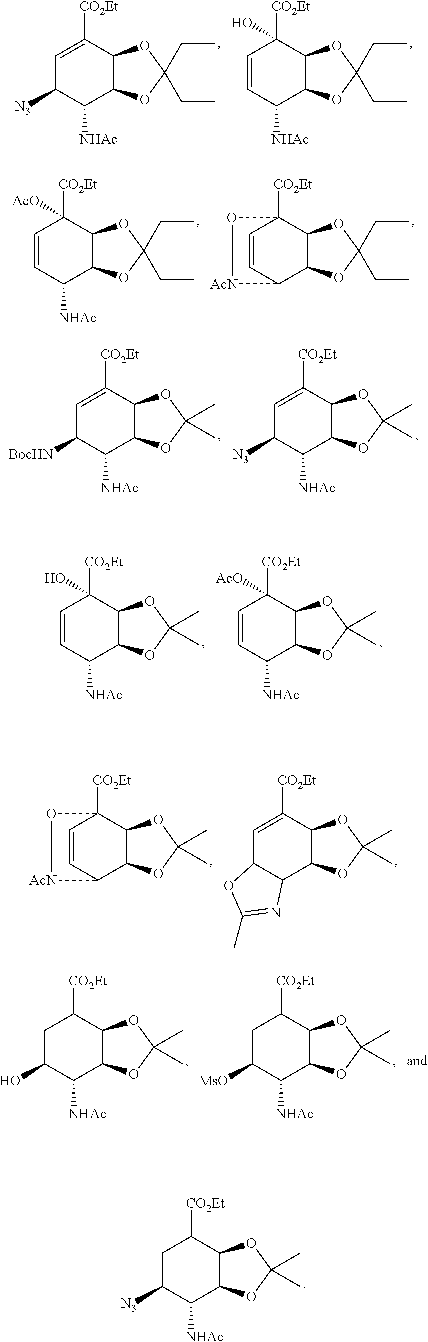

or salts, solvates, prodrugs, stereoisomers or istope-labelled forms thereof, or mixtures thereof.

### COMPOSITIONS AND THERAPEUTIC APPLICATIONS

As hereinbefore mentioned, novel compounds of the formulae A and B have been prepared. Accordingly, the present application includes all uses of the compounds of formulae A and B, including their use in therapeutic methods and compositions for treatment of influenza, their use in diagnostic assays and their use as research tools. In particular, the present application includes the use of a compound of formulae A and B as a medicament,

Another aspect of the application relates to a use of a compound of the application for the treatment or prevention of influenza.

Another aspect of the application relates to a use of a compound of the application for the preparation of a medicament for the treatment or prevention of influenza.

Also within the scope of the present application is a method of treating or preventing influenza comprising administering an effective amount of a compound of the application to a subject in need thereof.

The compounds of the application are suitably formulated into pharmaceutical compositions for administration to human subjects in a biologically compatible form suitable for administration in vivo. Accordingly, the present application further includes a pharmaceutical composition comprising a compound of the application and a pharmaceutically acceptable carrier and/or diluent.

The compositions containing the compounds of the application can be prepared by known methods for the preparation of pharmaceutically acceptable compositions, which can be administered to subjects, such that an effective quantity of the active substance is combined in a mixture with a pharmaceutically acceptable vehicle. Suitable vehicles are described, for example, in Remington's Pharmaceutical Sciences (2000-20th edition) and in The United States Pharmacopeia: The National Formulary (USP 24 NF19) published in 1999). On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of the application may be used in the form of the free base, in the form of salts and/or solvates. All forms are within the scope of the application.

In accordance with the methods of the application, the described compounds, salts or solvates thereof may be administered to a patient in a variety of forms depending on the selected route of administration, as will be understood by those skilled in the art. The compositions of the application may be administered, for example, by oral, parenteral, buccal, sublingual, nasal, rectal, patch, pump or transdermal (topical) administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound of the application may be incorporated with excipient and used in the form of ingestible tablets, buccal tablets, troches, capsules, elixirs, suspensions, syrups, wafers, and the like.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2000-20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists. Ampoules are convenient unit dosages.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve, which is intended for disposal after use. Where the dosage form comprises an aerosol dispenser, it will contain a propellant, which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

Compositions for topical administration may include, for example, propylene glycol, isopropyl alcohol, mineral oil and glycerin. Preparations suitable for topical administration include liquid or semi-liquid preparations such as liniments, lotions, applicants, oil-in-water or water-in-oil emulsions such as creams, ointments or pastes; or solutions or suspensions such as drops. In addition to the aforementioned ingredients, the topical preparations may include one or more additional ingredients such as diluents, buffers, flavouring agents, binders, surface active agents, thickeners, lubricants, preservatives, e.g. methyl hydroxybenzoate (including anti-oxidants), emulsifying agents and the like.

Sustained or direct release compositions can be formulated, e.g. liposomes or those wherein the active compound is protected with differentially degradable coatings, such as by microencapsulation, multiple coatings, etc. It is also possible to freeze-dry the compounds of the formula I and use the lypolizates obtained, for example, for the preparation of products for injection.

The dosage administered will vary depending on the use and known factors such as the pharmacodynamic characteristics of the particular substance, and its mode and route of administration; age, health, and weight of the individual recipient; nature and extent of symptoms, kind of concurrent treatment, frequency of treatment, and the effect desired.

The following non-limiting examples are illustrative of the present application:

### EXAMPLES

### Example 1

### N-(3aS,6S,7R,7aR)-(4,7-Dibromo-2,2-dimethyl-3a,4,5,7a-tetrahydro-benzo[1,3]dioxol-5-yl-acetamide (9)

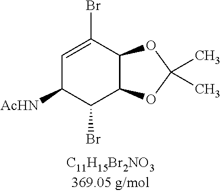

To a solution of N-bromoacetamide (309 mg, 2.25 mmol) in 40 mL acetonitrile was added 0.28 mL SnBr₄ (0.4 M in CH₂Cl₂, 0.11 mmol) at −40° C. in the dark. Diene 8 (432 mg; 1.87 mmol) in acetonitrile (20 mL) was added slowly to the reaction mixture by syringe pump at the same temperature over 4 h. The resulting reaction mixture was stirred for 1 h, before saturated aqueous NaHCO₃ (10 mL) and Na₂SO₃ (10 mL) were carefully added. The phases were separated and the aqueous phase was extracted with CH₂Cl₂ (3×100 mL). The combined organic extracts were washed with brine (10 mL), dried over MgSO₄ and concentrated under reduced pressure. The residue was purified by flash column chromatography on neutral alumina (CH₂Cl₂) to afford bromo amide 9 (526 mg, 76%) as colourless crystals: R_{f} 0.71 (CH₂Cl₂/MeOH 96:4); mp 181° C.; [α]_{D}²³ +188.2 (c 0.50, CHCl₃); IR (film) ν 3684, 3019, 2400, 1676, 1498, 1425, 1216, 1064, 929, 757, 669, 497, 478, 472 cm⁻¹; ¹H NMR (300 MHz, CDCl₃) δ 6.21 (d, J=9.0 Hz, 1H), 4.93 (m, 1H), 4.68 (d, J=5.1 Hz, 1H), 4.60 (t, J=4.5 Hz, 1H), 4.21 (t, J=3.6 Hz, 2H), 1.97 (s, 3H), 1.51 (s, 3H), 1.42 (s, 3H) ppm; ¹³C NMR (75 MHz, CDCl₃) δ 169.0, 128.0, 124.7, 112.0, 77.8, 75.8, 50.3, 44.4, 27.8, 26.5, 23.3 ppm; MS (EI) m/z (%): 366 (M), 354 (6), 313 (6), 294 (6), 255 (6), 253 (12), 251 (7), 232 (33), 230 (34), 190 (11), 189 (6), 188 (12), 187 (5), 174 (6), 173 (10), 172 (8), 171 (9), 165 (8), 163 (8), 151 (6), 109 (22), 108 (9), 93 (7), 81 (8), 80 (10), 65 (8), 59 (11), 55 (8), 43 (100), 42 (11), 41 (6); HRMS (EI) calcd for C₁₁H₁₅O₃NBr₂ 366.9419; found 366.9418.

### Example 2

### (1S,4S,5S,6S)-7-Acetyl-3-bromo-4,5-(isopropylidenedioxy)-7-azabicyclo[4.1.0]hept-2-ene (10)

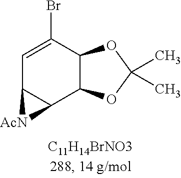

To a solution of amide 9 (17.61 g, 47.72 mmol) in dimethoxyethane (400 mL) was added n-Bu₄NBr (16.33 g, 52.49 mmol) at 0° C. under argon. At this temperature potassium bis(trimethylsilyl)amide (100 mL, 0.5 M in toluene, 52.49 mmol) was added dropwise. The reaction mixture was stirred for 3 h at 0° C. and then quenched by the addition of potassium phosphate monobasic sodium hydroxide (350 mL, set to pH 7). The two-phase mixture was extracted with ethyl acetate (3×150 mL) and then the combined organic phases were dried over MgSO₄ and concentrated under reduced pressure. The crude material was purified by flash column chromatography with a solvent gradient of 2:1 then 1:1 (hexanes-ethyl acetate) to afford (10) (9.21 g, 67%) as colourless crystals: R_{f} 0.44 (1:1, hexanes-ethyl acetate); mp 128° C.; [α]_{D}²³ −57.6 (c 0.75, CHCl₃); IR (film) ν 3017, 2938, 1704, 1423, 1383, 1372, 1289, 1267, 1216, 1161, 1063, 994, 967, 894, 868, 819, 756, 668, 619, 554, 510, 485, 468 cm⁻¹; ¹H NMR (300 MHz, CDCl₃) δ 6.70 (d, J=4.8 Hz, 1H), 4.72 (dd, J=0.9, 6.9 Hz, 1H), 4.46 (dd, J=4.2, 6.9 Hz, 1H), 3.19 (dd, J=5.1, 6.0 Hz, 1H), 3.11 (ddd, J=0.9, 6.0, 6.1 Hz, 1H), 2.17 (s, 3H), 1.56 (s, 3H), 1.41 (s, 3H) ppm; ¹³C NMR (75 MHz, CDCl₃) δ 181.7, 129.9, 122.8, 108.5, 76.6, 71.9, 39.6, 36.7, 27.0, 24.9, 23.2 ppm; MS (EI) m/z (%) 272 (M⁺-CH₃), 232 (9), 230 (9), 208 (5), 190 (9), 189 (8), 188 (9), 187 (7), 172 (6), 170 (5), 160 (13), 158 (13), 150 (18), 109 (18), 108 (45), 100 (23), 85 (10), 84 (5), 81 (9), 80 (17), 79 (7), 78 (7), 59 (9), 53 (7), 52 (6), 51 (10), 43 (100), 42 (6), 41 (6); HRMS (EI) calcd for C₁₁H₁₄O₃NBr 287.0157; found 287.0161.

### Example 3

### N1-[(3aS,4S,5R,7aS)-7-Bromo-2,2-dimethyl-5-(1-ethyl-propoxy)-3a,4,5,7a-tetrahydro-1,3-benzodioxol-4-yl]acetamide (11)

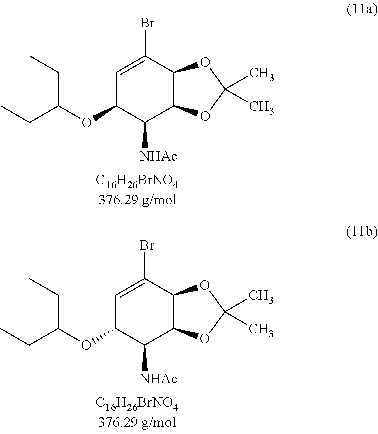

To a solution of aziridine 10 (7.78 g, 27.00 mmol) in 3-pentanol (30 mL) was added copper(II) trifluoromethanesulfonate (976 mg, 2.70 mmol) at 0° C. The reaction mixture was stirred for 16 h before concentrating under reduced pressure. The crude residue was dissolved in methylene chloride (20 mL), washed with sat. NaHCO₃ (3×5 mL), and brine (1×10 mL) and then dried over Na₂SO₄. The residue was purified by flash column chromatography with a solvent gradient of 3:1 then 1:1 (hexanes-diethyl ether) to afford 11a (1.09 g, 11%) and 11b (7.64 g, 76%) as clear oils.

(11a): R_{f} 0.45 (diethyl ether); [α]_{D}²³ +41.5 (c 1.0, CHCl₃); IR (film) ν 3439, 3019, 2971, 2936, 2879, 1675, 1514, 1463, 1384, 1373, 1342, 1514, 1463, 1384, 1373, 1342, 1216, 1163, 1095, 1046, 965, 931, 888, 865, 758, 669, 502 cm⁻¹; ¹H NMR (600 MHz, CDCl₃) δ 6.35 (d, J=8.7 Hz, 1NH), 6.31 (d, J=5.3 Hz, 1H), 4.57 (d, J=5.3 Hz, 1H), 4.40-4.44 (m, 2H), 3.90 (t, J=5.3 Hz, 1H), 3.19 (quin., J=5.8 Hz, 1H), 2.03 (s, 3H), 1.41-1.55 (m, 4H), 1.46 (s, 3H), 1.36 (s, 3H), 0.88 (t, J=7.6 Hz, 6H) ppm; ¹³C NMR (150 MHz, CDCl₃) δ 169.6, 129.0, 127.5, 111.1, 81.6, 76.7, 75.3, 70.6, 46.6, 27.5, 26.6, 26.4, 25.9, 23.3, 9.8, 9.6 ppm; MS (EI) m/z (%) 375 (M), 232 (8), 231 (7), 230 (9), 229 (5), 190 (21), 189 (16), 188 (22), 187 (13), 166 (6), 164 (6), 143 (22), 142 (100), 137 (9), 136 (8), 126 (6), 125 (6), 110 (5), 109 (32), 108 (7), 100 (30), 85 (6), 84 (61), 83 (9), 80 (9), 71 (8), 70 (9), 60 (9), 59 (12), 43 (73), 41 (7); HRMS (EI) calcd for C₁₆H₂₆BrNO₄ 375.1045; found 375.1045.

(11b): R_{f} 0.40 (diethyl ether); [α]_{D}²³ −100.0 (c 1.0, CHCl₃); IR (film) ν 3439, 3019, 2971, 2936, 2879, 1675, 1514, 1463, 1384, 1373, 1342, 1514, 1463, 1384, 1373, 1342, 1216, 1163, 1095, 1046, 965, 931, 888, 865, 758, 669, 502 cm⁻¹; ¹H NMR (600 MHz, CDCl₃) δ 6.14 (d, J=1.5 Hz, 1H), 5.83 (d, J=9.0 Hz, 1H), 4.57 (dd, J=1.5, 5.3 Hz, 1H), 4.40 (dd, J=2.4, 5.1 Hz, 1H), 4.30 (dt, J=2.4, 8.9 Hz, 1H), 3.89 (dd, J=1.5, 8.9 Hz, 1H), 3.24 (quin., J=5.6 Hz, 1H), 2.00 (s, 3H), 1.43-1.49 (m, 4H), 1.38 (s, 3H), 1.34 (s, 3H), 0.87 (t, J=7.4 Hz, 3H), 0.85 (t, J=7.4 Hz, 3H) ppm; ¹³C NMR (150 MHz, CDCl₃) δ 169.9, 132.5, 122.7, 110.2, 82.4, 77.3, 76.0, 73.6, 51.2, 27.4, 26.3, 26.1, 25.6, 23.5, 9.6, 9.2 ppm; MS (EI) m/z (%) 375 (M), 232 (8), 231 (7), 230 (9), 229 (5), 190 (21), 189 (16), 188 (22), 187 (13), 166 (6), 164 (6), 143 (22), 142 (100), 137 (9), 136 (8), 126 (6), 125 (6), 110 (5), 109 (32), 108 (7), 100 (30), 85 (6), 84 (61), 83 (9), 80 (9), 71 (8), 70 (9), 60 (9), 59 (12), 43 (73), 41 (7); HRMS (EI) calcd for C₁₆H₂₆BrNO₄ 375.1045; found 375.1045.

### Example 4

### N1-[(3aR,6R,7S,7aS)-7-Acetylamino-6-(1-ethyl-propoxy)-2,2-dimethyl-3a,6,7,7a-tetrahydro-benzo[1,3]dioxole-4-carboxylic acid ethyl ester (12)

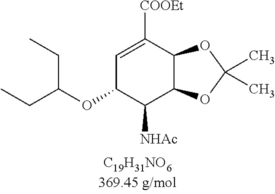

To a solution of vinylbromide 11b (6.50 g, 17.27 mmol) in toluene (300 mL) and ethanol (82 mL) was passed CO gas (1 atm). After 10 mins triethylamine (84.19 mL, 604.6 mmol) was added followed by tetrakis(triphenylphosphine)palladium(0) (998 mg, 0.864 mmol) at room temperature. The resulting solution was heated to 60° C. while a continuous flow of CO gas (1 atm) was passed. After 2 h dichlorobis(triphenylphosphine)palladium(II) (1.212 g, 1.727 mmol) was added in two portions over 15 mins. The reaction mixture was brought to reflux for 4 h, cooled to room temperature, and filtered through a plug of celite. The crude material was purified by flash column chromatography with a solvent gradient of 2:1 then 1:1 (hexanes-ethyl acetate) to yield (12) (4.34 g, 68%) as a colourless solid: R_{f} 0.22 (96:4, methylene chloride/methanol); mp 112-115° C.; [α]_{D}²³ −122.7 (c 1.0, CHCl₃); IR (film) ν 3383, 3022, 2975, 2879, 1711, 1663, 1576, 1464, 1374, 1254, 1218, 1094, 1068, 929, 776, cm⁻¹; ¹H NMR (600 MHz, CDCl₃) δ 6.90 (d, J=1.9 Hz, 1H), 5.82 (d, J=8.7 Hz, 1NH), 5.03 (dd, J=0.76, 5.7 Hz, 1H), 4.49 (dd, J=2.6, 5.7 Hz, 1H), 4.22-4.31 (m, 3H), 4.04 (d, J=9.0 Hz, 1H), 3.34 (quin, J=5.6 Hz, 1H), 2.03 (s, 3H), 1.48-1.56 (m, 4H), 1.37 (s, 3H), 1.34 (s, 3H), 1.30 (t, J=7.0 Hz, 3H), 0.92 (t, J=7.5 Hz, 3H), 0.89 (t, J=7.2 Hz, 3H) ppm; ¹³C NMR (150 MHz, CDCl₃) δ 169.9, 165.5, 141.1, 129.9, 109.5, 82.6, 74.7, 72.0, 71.6, 61.1, 51.9, 27.3, 26.1, 25.9, 25.5, 23.6, 14.2, 9.6, 9.3 ppm; MS (EI) m/z (%) 369 (M⁺-CH₃), 228 (10), 182 (12), 181 (11), 154 (6), 153 (8), 143 (8), 142 (88), 136 (10), 112 (8), 110 (7), 109 (7), 100 (17), 88 (6), 87 (13), 86 (32), 85 (8), 84 (100), 83 (9), 80 (6), 71 (12), 70 (11), 69 (5), 60 (8), 59 (13), 58 (5), 57 (11), 55 (11), 49 (10), 47 (11), 43 (87), 42 (6), 41 (13); HRMS (EI) calcd for C₁₈H₂₈NO₆ 354.1917; found 354.1919.

### Example 5

### (3R,4S,5S,6R)-4-Acetylamino-3-(1-ethyl-propoxy)-5,6-dihydroxy-cyclohex-1-enecarboxylic acid ethyl ester (13)

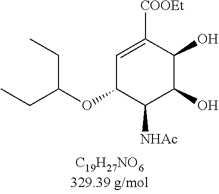

To a solution of ester 12 (1.13 g, 3.06 mmol) in ethanol (20 mL) was added 6 M HCL (500 μL) at room temperature. The resulting solution was stirred for 5 h at 60° C. before the addition of H₂O (500 μL). The reaction was stirred for an additional 1 h at 60° C. before cooling to room temperature and concentrating under reduced pressure. The crude material was purified by flash column chromatography with a solvent system of 24:1 (methylene chloride/methanol) to yield (13) (715 mg, 71%) as white solid: R_{f} 0.71 (9:1, methylene chloride/methanol); [α]_{D}²³ −44.1 (c 0.47, CHCl₃); mp 154° C.; IR (film) ν 3380, 3020, 2970, 2937, 2879, 1715, 1661, 1576, 1464, 1374, 1244, 1217, 1094, 1060, 929, 756, 667 cm⁻¹; ¹H NMR (600 MHz, (CD₃)₂CO) δ 7.36 (d, J=6.4 Hz, 1NH), 6.78 (d, J=3.8 Hz, 1H), 4.86 (d, J=4.2 Hz, 10H), 4.64 (t, J=3.9 Hz, 1H), 4.21-4.25 (m, 1H) 4.21 (dq, J=1.1, 7.2 Hz, 2H), 4.01-4.15 (m, 2H), 3.96 (dd, J=4.3, 6.6 Hz, 1H), 3.56 (quin, J=5.8 Hz, 1H), 1.87 (s, 3H), 1.44-1.58 (m, 4H), 1.28 (t, J=7.2 Hz, 3H), 0.91 (t, J=7.6 Hz, 3H), 0.87 (t, J=7.6 Hz, 3H) ppm; ¹³C NMR (150 MHz, (CD₃)₂CO) δ 169.2, 165.9, 137.7, 132.6, 81.4, 73.4, 67.0, 65.7, 60.4, 53.1, 26.1, 26.0, 22.4, 13.6, 9.2, 8.8 ppm; MS (FAB) m/z (%) 330 (M⁺), 260 (21), 242 (25), 224 (13), 182 (23), 178 (14), 152 (11), 136 (13), 112 (11), 110 (21), 109 (16), 81 (13), 71 (13), 69 (17), 67 (10), 60 (19), 57 (19), 55 (31), 43 (100), 41 (34), 39 (16), 29 (45); HRMS calcd for C₁₆H₂₈NO₆⁺330.1917; found 330.1919.

### Example 6

### (3R,4S,5S,6R)-4-Acetylamino-3-(1-ethyl-propoxy)-6-hydroxy-5-(toluene-4-sulfonyloxy)-cyclohex-1-enecarboxylic acid ethyl ester (14)

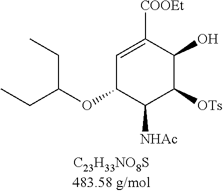

To a solution of diol 13 (2.32 g, 7.04 mmol) in dry pyridine (15 mL) was added 4-toluenesulfonyl chloride (1.48 g, 7.74 mmol) portion wise over 5 mins. The reaction mixture was stirred at room temperature for 48 h and then diluted with methylene chloride (10 mL). The organic layer was washed with cold 1N HCl (3×5 mL), brine (1×10 mL) and dried over Na₂SO₄. The crude mixture was purified by flash column chromatography with a solvent system gradient of 99:1 then 96:4 (methylene chloride/methanol) to yield (14) (1.76 g, 76%, based on 0.587 g recovered starting material) as clear oil; R_{f} 0.71 (96:4, methylene chloride/methanol); [α]_{D}²³ −36.6 (c 2.5, CHCl₃); IR (film) ν 3375, 2971, 2937, 2879, 2733, 2458, 2252, 1920, 1716, 1660, 1598, 1527, 1463, 1444, 1372, 1248, 1218, 1190, 1178, 1121, 1096, 1059, 1002, 970, 915, 848, 815, 769, 704, 666, 556, 486 cm⁻¹; ¹H NMR (600 MHz, CDCl₃) δ 7.81 (d, J=8.3 Hz, 2H), 7.35 (d, J=8.3 Hz, 2H), 7.14 (d, J=6.8 Hz, 1NH), 6.87 (d, J=4.9 Hz, 1H), 4.95 (t, J=3.5 Hz, 1H), 4.76 (t, J=3.8 Hz, 1H), 4.26 (q, J=7.2 Hz, 2H), 4.15 (dt, J=3.2, 6.8 Hz, 1H), 4.08 (dd, J=2.6, 4.9 Hz, 1H), 3.45 (quin, J=5.8 Hz, 1H), 3.25 (d, J=3.4 Hz, 10H), 2.45 (s, 3H), 1.92 (s, 3H), 1.45-1.51 (m, 2H), 1.34-1.40 (m, 1H), 1.32 (t, J=7.2 Hz, 3H), 1.24-1.26 (m, 1H), 0.84 (t, J=7.4, 3H), 0.70 (t, J=7.2 Hz, 3H) ppm; ¹³C NMR (150 MHz, CDCl₃) δ 170.3, 165.3, 145.4, 137.4, 132.9, 131.2, 130.1 (2×C), 128.0 (2×C), 82.2, 74.6, 72.6, 64.6, 61.6, 50.9, 26.3, 25.9, 23.4, 21.7, 14.2, 9.8, 8.9 ppm; MS (FAB) m/z (%) 484 (M⁺), 29 (13), 39 (10), 41 (13), 43 (27), 55 (12), 57 (8), 69 (6), 77 (5), 91 (8), 136 (8), 178 (5), 224 (7), 396 (5); HRMS (FAB) calcd for C₂₃H₃₄NO₈S 484.2005; found 484.1998.

### Example 7

### (3R,4S,5S,6R)-4-Acetylamino-5-azido-3-(1-ethyl-propoxy)-6-hydroxy-cyclohex-1-enecarboxylic acid ethyl ester (15)

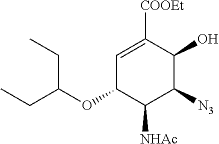

To a stirred solution of tosylate (14) (24 mg, 0.049 mmol) in N,N-dimethylformamide (2 mL) was added tetrabutylammonium azide (141 mg, 0.49 mmol). The resulting suspension was heat to reflux for 16 h, cooled and concentrated under reduced pressure. The crude material was purified by flash column chromatography with a solvent system of 2:1 (hexanes-ethyl acetate) to yield (15) (12 mg, 69%) as a pale yellow oil: R_{f} 0.27 (1:1 hexanes-ethyl acetate); [α]_{D}²³ −39.71 (c 1.00, CHCl₃); mp 112-115° C. (hexanes-ethyl acetate); IR (film) ν 3382, 2966, 2927, 2863, 1719, 1653 cm⁻¹; ¹H NMR (600 MHz, CDCl₃) δ 6.87 (dd, J=0.94, 2.8 Hz, 1H), 6.10 (d, J=7.9 Hz, 1NH), 4.71 (ddd, J=0.94, 1.1, 6.4 Hz, 1H), 4.48 (dd, J=6.4, 9.4 Hz, 1H), 4.43 (ddd, J=2.1, 2.7, 6.8 Hz, 1H), 4.28 (dq, J=2.7, 7.1 Hz, 2H), 3.82 (ddd, J=6.7, 7.9, 9.4 Hz, 1H), 3.47 (quin, J=5.8 Hz, 1H), 2.01 (s, 3H), 1.48-1.56 (m, 4H), 1.33 (t, J=7.2 Hz, 3H), 0.93 (t, J=7.5 Hz, 3H), 0.91 (t, J=7.5 Hz, 3H) ppm; ¹³C NMR (150 MHz, CDCl₃) δ 170.2, 166.0, 139.4, 130.3, 82.5, 73.0, 71.3, 61.5, 58.9, 55.9, 26.1, 25.8, 23.5, 14.2, 9.6, 9.4 ppm; MS (FAB) m/z (%) 348 (M⁺-7), 29 (57), 43 (100), 77 (27), 107 (16), 136 (26), 172 (15), 224 (23), 242 (31), 260 (64), 278 (26).

### Example 8

### (3R,4S,5S,6R)-4-Acetylamino-5-azido-3-(1-ethyl-propoxy)-6-methylsulfonyloxy-cyclohex-1-enecarboxylic acid ethyl ester (16)

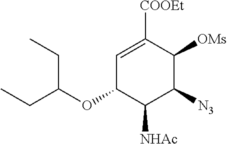

To a stirred solution of alcohol 15 (23 mg, 0.065 mmol) in methylene chloride (1 mL) was added triethylamine (72 μL, 0.52 mmol). The resulting solution was cooled to −78° C. prior to the addition of methanesulfonyl chloride (15 μL, 0.19 mmol). The reaction mixture was allowed to warm to 15° C. slowly over 3 h, quenched by the addition of sat. NaHCO₃ (1 mL), extracted into methylene chloride (3×1 mL) and dried over Na₂SO₄. The crude material was purified by flash column chromatography with a solvent gradient of 2:1, then 1:1 (hexanes-ethyl acetate) to yield (16) (21 mg, 75%) as a white solid: R_{f} 0.40 (1:1 hexanes-ethyl acetate); [α]_{D}²³ −3.88 (c 0.50, CHCl₃); mp 96-98° C.; IR (film) ν 3345, 2968, 2932, 2877, 1717, 1655, 1558, 1362, 1264 cm⁻¹; ¹H NMR (600 MHz, CDCl₃) δ 7.15 (d, J=3.4 Hz, 1H), 6.14 (d, J=8.5 Hz, 1NH), 5.65 (d, J=4.14 Hz, 1H), 4.73 (dd, J=4.2, 5.8 Hz, 1H), 4.26-4.31 (m, 3H), 4.22 (ddd, J=3.4, 5.3, 8.5 Hz, 1H), 3.58-3.62 (m, 1H), 3.18 (s, 3H), 2.00 (s, 3H), 1.49-1.56 (m, 4H), 1.34 (t, J=7.1 Hz, 3H), 0.93 (t, J=7.3 Hz, 3H), 0.91 (t, J=7.4 Hz, 3H) ppm; ¹³C NMR (150 MHz, CDCl₃) δ 169.7, 164.4, 143.1, 125.5, 82.5, 72.4, 65.8, 61.9, 54.5, 53.4, 38.9, 26.1, 25.8, 23.4, 14.1, 9.9, 9.2 ppm; MS (EI) m/z (%) 425 (M⁺-7), 43 (100), 55 (11), 79 (12), 120 (15), 136 (37), 152 (29), 201 (40), 224 (21), 242 (79), 300 (17), 329 (7), 360 (5).

### Example 9

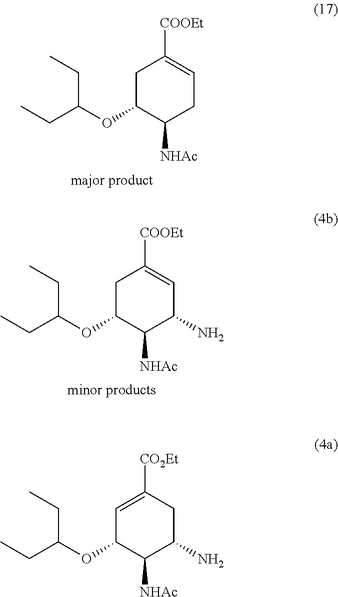

Compound 16 (32 mg, 0.07 mmol) was dissolved in 0.5 mL ethanol and 0.1 mL THF and cooled to −30° C. Sodium borohydride (14 mg, 0.37 mmol) was added in small portions. The reaction was stirred for 5 h at −30° C. and slowly warmed to room temperature before it was stirred for another 12 h, concentrated at reduced pressure, and partitioned between CH₂Cl₂ and water. The organic phase was dried over MgSO₄, filtered and evaporated. Purification by flash column chromatography (CH₂Cl₂/MeOH 96:4 to 4:1) afforded compound 17 as the major product and a mixture of oseltamivir isomers 4a and 4b.

4a and 4b mixture: R_{f} 0.40 (CH₂Cl₂/MeOH 8:2); ¹H NMR (600 MHz, CDCl₃) δ=7.14 (d, J=5.1 Hz, 1H), 6.83 (d, J=5.1 Hz, 1H), 5.45 (bs, N—H), 4.15 (q, J=7.2 Hz, 2H), 3.83 (m, 1H), 3.68 (m, 1H), 3.39 (quint., J=5.7 Hz, 1H), 2.87 (dd, J=5.1 Hz, J=17.1 Hz, 1H), 2.25 (m, 1H), 1.96 (s, 3H), 1.40 (m, 4H), 1.19 (t, J=7.2 Hz, 3H), 0.76 (t, J=7.2 Hz, 3H), 0.75 (t, J=7.2 Hz, 3H) ppm.

### Example 10

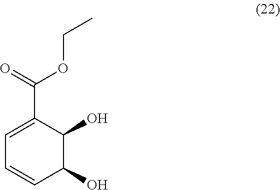

White solid; mp 48° C. (ethyl acetate-hexanes); R_{f} 0.31 (1:2 hexanes-ethyl acetate); [α]_{D}²³ +54.7 (c 3.75, CHCl₃); IR (film) ν 3385, 2981, 2934, 1700, 1280, 1243, 1104, 1068, 825, 771 cm⁻¹; ¹H NMR (300 MHz, CDCl₃) δ 7.04 (d, J=5.3 Hz, 1H), 6.15 (dt, J=1.1, 9.4 Hz, 1H), 6.03 (dq, J=2.25, 9.22 Hz, 1H), 4.49-4.55 (m, 1H), 4.40-4.48 (m, 1H), 4.22 (q, J=7.0 Hz, 2H), 3.65-3.78 (m, 2H), 1.28 (t, J=7.2 Hz, 3H) ppm; ¹³C NMR (75 MHz, CDCl₃) δ 167.1, 138.7, 134.1, 128.7, 122.5, 69.8, 64.5, 60.9, 14.2 ppm; MS (EI) m/z (%) 184 (M), 45 (20), 51 (21), 77 (39), 105 (100), 121 (52), 122 (33), 138 (26), 166 (20); HRMS calcd for C₉H₁₂O₄ 184.0736; found 184.0731; Anal. calcd: C, 58.69, H, 6.57; found C, 58.77, H, 6.60.

### Example 11

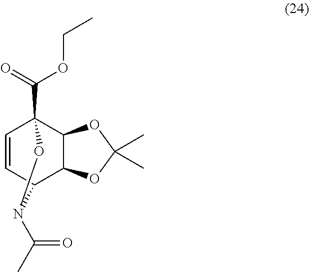

To a stirred solution of 22 (5 g, 27.1 mmol) in 2,2-dimethoxypropane (80 mL) was added p-toluenesulfonic acid (catalytic amount) at room temperature. After complete consumption of starting material (TLC analysis), the solution was cooled 0° C. before the addition of H₂O (10 mL). On a preparative scale the intermediate acetonide was not isolated (analytical samples were purified via flash column chromatography with a solvent system of 3:1 (hexanes-ethyl acetate)). Data for the intermediate 23: R_{f} 0.56 (1:1 hexanes/ethyl acetate); [α]_{D}²³ +74.6 (c 4.02, CHCl₃); IR (film): ν 3018, 2987, 2936, 1712, 1651, 1425, 1380, 1259, 1155, 1031, 917, 856, 697, 667, 512 cm⁻¹; ¹H NMR (300 MHz, CDCl₃) δ 7.06 (dd, J=5.3, J=3.1 Hz, 1H), 4.84 (d, J=5.7 Hz, 1H), 4.28-4.41 (m, 1H), 4.07-4.26 (m, 2H), 2.21-2.45 (m, 1H), 1.99-2.16 (m, 1H), 1.86-1.99 (m, 1H), 1.58-1.72 (m, 1H), 1.33 (d, J=10.2 Hz, 6H), 1.24 (t, J=7.2 Hz, 3H) ppm; ¹³C NMR (75 MHz, CDCl₃) δ 166.2, 142.3, 130.0, 108.5, 72.6, 70.4, 60.5, 27.8, 26.2, 25.1, 20.9, 14.2 ppm; MS (EI) m/z (%): 226 (M⁺-CH₃), 211 (77), 181 (15), 169 (17), 123 (100), 105 (17), 95 (13), 83 (11), 79 (76), 67 (14), 59 (10), 55 (11), 43 (82), 41 (14); HRMS (M⁺-CH₃) calcd for C₁₂H₁₆O₄ 211.0970; found 211.0969; Anal. calcd: C, 64.27; H, 7.19. Found C, 64.52; H, 7.08.

NalO₄ (5.80 g, 27.1 mmol) was added to the reaction vessel prior to the addition of a solution of acetohydroxamic acid (2.03 g, 27.1 mmol) in MeOH (25 mL) dropwise over 5 minutes. The resulting solution was stirred at room temperature for 16 h, quenched by the slow addition of sat. NaHSO₃ (10 mL) and extracted into Et₂O (3×100 mL). The combined organic layers were washed with brine (2×30 mL) and dried over Na₂SO₄. The crude material was purified via flash column chromatography with a solvent system of 2:8 (hexanes-ethyl acetate) to yield 24 (5.65 g, 70% over 2 steps) as a white solid: R_{f} 0.33 (3:7 hexanes-ethyl acetate); mp 89-90° C. (hexanes-ethyl acetate); [α]_{D}²³ −18.0 (c 0.54, CHCl₃); IR (film) ν 3466, 2938, 2987, 1747, 1684, 1620, 1372, 1275, 1086 cm⁻¹; ¹H NMR (600 MHz, CDCl₃) δ 6.57-6.65 (m, 2H), 5.47-5.52 (m, 1H), 4.71 (d, J=6.8 Hz, 1H), 4.56 (dd, J=4.7, 6.6 Hz, 1H), 4.38 (q, J=7.2 Hz, 2H), 2.01 (s, 3H), 1.38 (t, J=7.2 Hz, 3H), 1.32 (s, 3H), 1.30 (s, 3H) ppm; ¹³C NMR (150 MHz, CDCl₃) δ 173.9, 166.6, 132.4, 128.4, 111.7, 79.2, 76.1, 72.8, 62.7, 50.0, 25.6, 25.4, 21.7, 14.1 ppm; MS (EI) m/z (%): 297 (M), 43 (100), 96 (30), 100 (32), 105 (35), 124 (52); HRMS calcd for C₁₄H₁₉NO₆ 297.1212; found 297.1215.

### Example 12

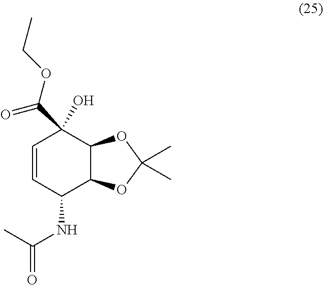

To a stirred solution of 24 (955 mg, 3.21 mmol) in 15:1/CH₃CN:H₂O (10 mL) was added molybdenum hexacarbonyl (848 mg, 3.21 mmol) at room temperature. The reaction was brought to reflux for 3 h, and then cooled to room temperature before the addition of activated charcoal (spatula tip). The resulting suspension was stirred for 30 mins and then filtered through a plug of celite. The crude material was purified via flash column chromatography with a solvent system of 1:9 (hexanes-ethyl acetate) to yield 25 (720 mg, 75%) as a white solid: R_{f} 0.20 (ethyl acetate); mp 97-99° C. (hexanes-ethyl acetate); [α]_{D}²³ −94.3 (c 0.79, CHCl₃); IR (film) ν 3433, 2094, 1644, 1271, 1217, 1060 cm⁻¹; ¹H NMR (600 MHz, CDCl₃) δ 6.25 (d, J=8.7 Hz, 1NH), 5.98 (dd, J=3.8, 9.8 Hz, 1H), 5.94 (dd, J=0.9, 9.9 Hz, 1H), 4.77-4.81 (m, 1H), 4.37 (t, J=8.3 Hz, 1H), 4.34 (dd, J=4.3, 7.7 Hz, 1H), 4.22-4.29 (m, 2H), 4.12 (s, 10H), 1.99 (s, 3H), 1.35 (s, 3H), 1.32 (t, J=7.4 Hz, 3H), 1.28 (s, 3H) ppm; ¹³C NMR (150 MHz, CDCl₃) δ 172.7, 170.0, 132.9, 129.6, 109.3, 81.0, 76.3, 74.5, 62.8, 48.8, 26.2, 24.2, 23.5, 14.0 ppm; MS (EI) m/z (%): 284 (M-CH₃⁺), 43 (90), 83 (47), 84 (100), 86 (61), 96 (37), 125 (36), 153 (38), 199 (99); HRMS calcd for C₁₃H₁₈NO₆284.1130; found 284.1137.

### Example 13

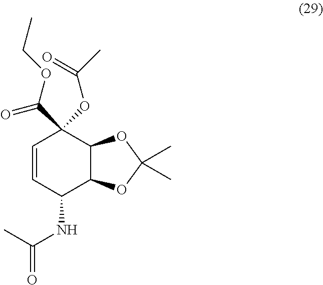

To a stirred solution of 25 (527 mg, 1.76 mmol) in acetic anhydride (3 mL) was added DMAP (catalytic amount) at room temperature. The resulting solution was stirred for 1 h before diluting with EtOAc (2 mL) and H₂O (2 mL). The reaction was quenched by the slow addition of NaHCO₃, and then extracted into EtOAc (3×2 mL). The combined organic layers were washed with brine (1×2 mL), dried over NaSO₄ and concentrated under reduced pressure. The crude material was purified via flash column chromatography with a solvent system of 1:9 (hexanes-ethyl acetate) to yield 29 (565 mg, 94%) as a white solid: R_{f} 0.26 (2:8 hexanes-ethyl acetate); mp 189-190° C. (hexanes-ethyl acetate); [α]_{D}²³+69.4 (c 0.27, CHCl₃); IR (film) ν 3429, 3264, 2916, 1758, 1630, 1374, 1226, 1050 cm⁻¹; ¹H NMR (600 MHz, CDCl₃) δ 6.59 (d, J=10.2 Hz, 1H), 6.14 (dd, J=5.9, 10.0 Hz, 1H), 5.52 (d, J=8.7 Hz, 1NH), 4.79 (ddd, J=2.7, 5.8, 8.8 Hz, 1H), 4.64 (dd, J=0.76, 6.8 Hz, 1H), 4.51 (dd, J=2.6, 6.8 Hz, 1H), 4.21-4.31 (m, 2H), 2.10 (s, 3H), 1.99 (s, 3H), 1.34 (s, 3H), 1.27-1.30 (m, 6H) ppm; ¹³C NMR (150 MHz, CDCl₃) δ 169.3, 168.5, 167.8, 131.9, 128.5, 109.5, 78.0, 77.1, 75.7, 62.0, 46.9, 26.1, 24.5, 23.6, 21.0, 14.0 ppm; MS (EI) m/z (%): 326 (M-CH₃⁺), 43 (100), 136 (12), 153 (13), 199 (17), 282 (10); HRMS calcd for C₁₅H₂₀NO₇ 326.1240; found 326.1235.

### Example 14

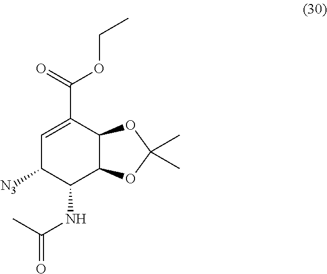

A solution of 29 (102 mg, 0.299 mmol) and trimethylsilyl azide (138 mg, 1.20 mmol) in THF (1 mL) was brought to reflux prior to the addition of freshly prepared tetrakis(triphenylphosphine)palladium (17 mg, 0.0149 mmol) in one portion. The reaction was allowed to reflux for 1 h, then cooled to room temperature and concentrated under reduced pressure. The crude material was purified via flash column chromatography with a solvent system of 7:3 (hexanes-ethyl acetate) to yield 30 (75 mg, 77%) as a pale yellow solid: R_{f} 0.51 (8:2 hexanes-ethyl acetate); mp 156-157° C. (CHCl₃); [α]_{D}²³ −183.6 (c 0.34, CHCl₃); IR (film) ν 3583, 3284, 2987, 2108, 1720, 1655, 1540, 1372, 1248, 1072 cm⁻¹; ¹H NMR (600 MHz, CDCl₃) δ 6.97 (d, J=4.5 Hz, 1H), 5.64 (br s, 1NH), 4.96 (d, J=5.7 Hz, 1H), 4.54 (t, J=4.3 Hz, 1H), 4.44 (td, J=4.4, 7.9 Hz, 1H), 4.25-4.32 (m, 3H), 2.04 (s, 3H), 1.43 (s, 3H), 1.41 (s, 3H), 1.33 (t, J=7.2 Hz, 3H) ppm; ¹³C NMR (150 MHz, CDCl₃) δ 170.8, 164.7, 136.2, 132.0, 110.4, 73.1, 70.1, 61.6, 56.7, 50.2, 27.7, 25.9, 23.3, 14.2 ppm; MS (EI) m/z (%): 309 (M-CH₃⁺), 43 (100), 84 (37), 167 (15), 224 (10), 309 (18); HRMS calcd for C₁₃H₁₇N₄O₅ 309.1200; found 309.1197.

### Example 15

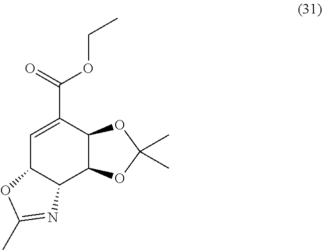

To a stirred solution of 25 (400 mg, 1.33 mmol) in methylene chloride (5 mL) was added NEt₃ (0.74 mL, 4.0 mmol), DMAP (catalytic amount) and mesityl chloride (0.16 mL, 1.4 mmol) at room temperature. The resulting solution was stirred for 4 h before being quenched by the slow addition of sat. NaHCO₃ (5 mL), and then extracted into ethyl acetate (3×5 mL). The combined organic layers were washed with brine (1×2 mL), dried over NaSO₄ and concentrated under reduced pressure. The crude material was purified via flash column chromatography with a solvent system of 1:2 (hexanes-ethyl acetate) to yield 31 (204 mg, 54%) as a white yellow solid: R_{f} 0.40 (1:4 hexanes-ethyl acetate); mp 54-55° C. (hexanes-ethyl acetate); [α]_{D}²³+150.4 (c 1.25, CHCl₃); IR (film) ν 3543, 2986, 1722, 1667, 1372, 1218 cm⁻¹; ¹H NMR (600 MHz, CDCl₃) δ 6.56 (d, J=3.0 Hz, 1H), 5.14 (dd, J=2.9, 8.5 Hz, 1H), 4.93 (d, J=5.2 Hz, 1H), 4.86 (dd, J=2.7, 5.1, Hz, 1H), 4.58 (d, J=8.4 Hz, 1H), 4.27-4.34 (m, 2H), 1.97 (d, J=1.3 Hz, 3H), 1.42 (s, 3H), 1.34 (t, J=7.2 Hz, 3H), 1.32 (s, 3H) ppm; ¹³C NMR (150 MHz, CDCl₃) δ 165.9, 165.6, 133.3, 130.5, 109.1, 73.6, 73.2, 68.9, 64.3, 61.2, 27.8, 26.3, 14.2, 14.1 ppm; MS (EI) m/z (%): 266 (M-CH₃⁺), 43 (52), 136 (19), 266 (100); HRMS calcd for C₁₃H₁₆NO₅266.1028; found 266.1032.

### Example 16

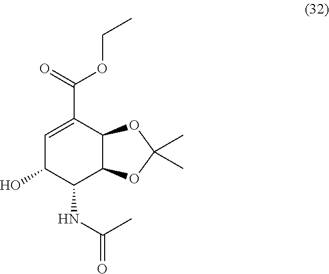

To a stirred solution of 31 (400 mg, 1.33 mmol) in methylene chloride (5 mL) was added NEt₃ (0.74 mL, 4.0 mmol), DMAP (catalytic amount) and mesityl chloride (0.16 mL, 1.4 mmol) at room temperature. The resulting solution was stirred for 4 h before being quenched by the slow addition of sat. NaHCO₃ (5 mL), and then extracted into ethyl acetate (3×5 mL). The combined organic layers were washed with brine (1×2 mL), dried over NaSO₄ and concentrated under reduced pressure. The crude material was purified via flash column chromatography with a solvent system of 1:2 (hexanes-ethyl acetate) to yield 32 (204 mg, 54%) as a white yellow solid: R_{f} 0.40 (1:4 hexanes-ethyl acetate); mp 54-55° C. (hexanes-ethyl acetate); [Q]_{D}²³+150.4 (c 1.25, CHCl₃); IR (film) ν 3543, 2986, 1722, 1667, 1372, 1218 cm⁻¹; ¹H NMR (600 MHz, CDCl₃) δ 6.56 (d, J=3.0 Hz, 1H), 5.14 (dd, J=2.9, 8.5 Hz, 1H), 4.93 (d, J=5.2 Hz, 1H), 4.86 (dd, J=2.7, 5.1, Hz, 1H), 4.58 (d, J=8.4 Hz, 1H), 4.27-4.34 (m, 2H), 1.97 (d, J=1.3 Hz, 3H), 1.42 (s, 3H), 1.34 (t, J=7.2 Hz, 3H), 1.32 (s, 3H) ppm; ¹³C NMR (150 MHz, CDCl₃) δ 165.9, 165.6, 133.3, 130.5, 109.1, 73.6, 73.2, 68.9, 64.3, 61.2, 27.8, 26.3, 14.2, 14.1 ppm; MS (EI) m/z (%): 266 (M-CH₃⁺), 43 (52), 136 (19), 266 (100); HRMS calcd for C₁₃H₁₆NO₅266.1028; found 266.1032.

### Example 17

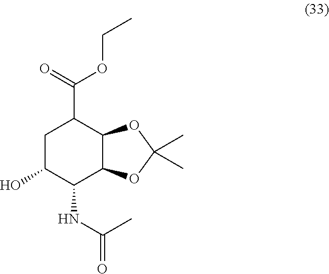

A hydrogenation vial was charged with 32 (150 mg, 0.501 mmol), 5% Rh/Al₂O₃ (60 mg) and 85% ethanol (2 mL) before evacuating with H₂. The reaction was stirred at room temperature and 60 psi for 144 h before filtering through a plug of SiO₂ and concentrating. The crude material was purified via flash column chromatography with a solvent gradient of 1:1 (hexanes-ethyl acetate) then methanol to yield 33 (143 mg, 95%) as a white solid: R_{f} 0.66 (90:10 CHCl₃-methanol); mp 156-158° C. (CHCl₃); [α]_{D}²³ −90.15 (c 1.1, CHCl₃); IR (film) ν 3305, 2986, 1722, 1666, 1553, 1374, 1219, 1066, 771 cm⁻¹; ¹H NMR (600 MHz, (CO(CD₃)₂) δ 7.17 (d, J=7.7 Hz, 1NH), 4.59 (t, J=4.1 Hz, 1H), 4.38 (br s, 10H), 4.21 (dq, J=7.2, 10.9 Hz, 1H), 4.14 (dd, J=4.6, 9.0 Hz, 1H), 4.11 (dq, J=3.3, 7.0 Hz, 1H), 4.00-4.03 (m, 1H), 3.85 (td, J=2.2, 4.5 Hz, 1H), 3.24 (dt, J=4.3, 12.6 Hz, 1H), 1.92 (s, 3H), 1.88-2.00 (m, 2H), 1.42 (s, 3H), 1.28 (s, 3H), 1.24 (t, J=7.2 Hz, 3H) ppm; MS (EI) m/z (%): 301 (M), 43 (38), 47 (29), 49 (24), 47 (100), 86 (80), 91 (20); HRMS calcd for C₁₄H₂₃—NO₆ 301.1525; found 301.1524.

### Example 18

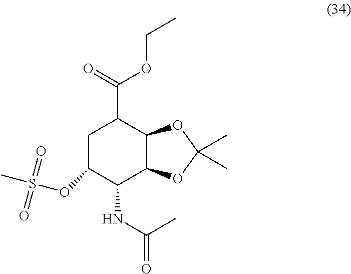

To a stirred solution of alcohol 33 (50 mg, 0.17 mmol) and triethylamine (93 μL, 0.66 mmol) in methylene chloride (100 μL) was added methanesulfonic anhydride (58 mg, 0.33 mmol) at 0° C. The reaction was slowly raised to room temperature over 24 h before it was diluted with methylene chloride (500 μL), and then washed with 1N HCl (2×500 μL), sat. NaHCO₃ (2×500 μL) and brine (1×1 mL). The crude material was purified via flash column chromatography with a solvent gradient of 1:2 then 1:5 (hexanes-ethyl acetate) to yield 34 (46 mg, 73%) as a white solid: R_{f} 0.47 (1:10 hexanes-ethyl acetate); mp 101-102° C. (CHCl₃); [α]_{D}²³ −49.91 (c 1.21, CHCl₃); IR (film) ν 3307, 2628, 1719, 1651, 1361 cm cm⁻¹; ¹H NMR (600 MHz, CDCl₃) δ 5.15-5.17 (m, 1H), 4.74 (d, J=8.9 Hz, 1NH), 4.62 (t, J=4.1 Hz, 1H), 4.22-4.28 (m, 1H), 4.15-4.19 (m, 1H), 4.03 (dd, J=4.6, 8.6 Hz, 1H), 3.58 (dt, J=2.7, 8.8 Hz, 1H), 3.12 (s, 3H), 2.92 (dt, J=4.2, 13.3 Hz, 1H), 2.25 (dt, J=4.2, 14.9 Hz, 1H), 2.09-2.13 (m, 1H), 2.08 (s, 3H), 1.55 (s, 3H), 1.37 (s, 3H), 1.26 (t, J=7.1 Hz, 3H) ppm; ¹³C NMR (150 MHz, CDCl₃) δ 170.6, 169.7, 109.9, 77.7, 73.8, 73.3, 61.1, 56.4, 42.6, 37.6, 28.1, 26.2, 25.2, 21.0, 14.1 ppm; MS (FAB) m/z (%): 380 (M+H⁺), 43 (23), 257 (100); HRMS calcd for C₁₅H₂₆NO₈S⁺380.1379; found 380.1366.

### Example 19

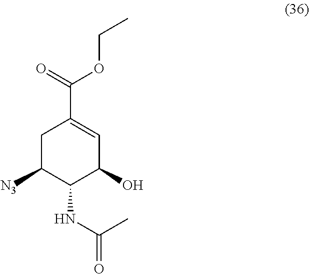

To a stirred solution of 34 (25 mg, 0.066 mmol) in acetone: H₂O/10:1 (×mL) was added sodium azide (mg, mmol). The resulting solution was stirred at room temperature for 12 h and then concentrated under reduced pressure. The resulting azide was used without further purification; data for 35: R_{f} 0.41 (1:10 hexanes-ethyl acetate); ¹H NMR (300 MHz, CDCl₃) δ 5.77 (d, J=7.5 Hz, 1NH), 4.58 (dd, J=4.0, 4.8 Hz, 1H), 4.43 (dd, J=4.8, 8.6 Hz, 1H), 4.13-4.29 (m, 2H), 3.91 (dt, J=3.4, 11.7 Hz, 1H), 3.20 (dd, J=8.1, 11.1 Hz, 1H), 2.85 (dt, J=3.9, 13.2 Hz, 1H), 2.17 (dt, J=3.9, 13.2 Hz, 1H), 2.02 (s, 3H), 1.91 (q, J=13.1 Hz, 1H), 1.51 (s, 3H), 1.34 (s, 3H), 1.26 (s, 3H) ppm; MS (FAB) m/z (%): 327 (M+Hⁱ), 43 (23), 257 (100), 299 (33); HRMS calcd for C₁₄H₂₃N₄O₅ 327.1668; found 327.1670. To a stirred solution of crude azide in methylene chloride (150 μL) was added 1,8-diazabicyclo[5.4.0]undec-7-ene (14.8 μL, 0.099 mmol) at 0° C. The resulting solution was stirred until complete consumption of starting material (TLC analysis, ˜12 h). The reaction was diluted with methylene chloride (500 μL), washed with 1N HCl (3×250 μL), and brine (1×500 μL) and then dried over Na₂SO₄. The crude material was purified by flash column chromatography with a solvent system of 1:7 (hexanes-ethyl acetate) to yield 36 (15 mg, 86% over 2 steps) as a yellow oil: R_{f} 0.22 (1:10 hexanes-ethyl acetate); IR (film) ν 3409, 2103, 1701, 1550, 1310, 1214 cm⁻¹, ¹H NMR (600 MHz, CDCl₃) δ 6.83 (t, J=2.4 Hz, 1H), 5.96 (br s, 1NH), 5.28 (br s, 10H), 4.39 (d, J=2.8 Hz, 1H), 4.22 (q, J=7.2 Hz, 2H), 3.59-3.66 (m, 2H), 2.98 (dd, J=4.3, 15.9 Hz, 1H), 2.42-2.49 (m, 1H), 2.10 (s, 3H), 1.30 (t, J=7.2 Hz, 3H) ppm; ¹³C NMR (150 MHz, CDCl₃) δ 173.6, 165.6, 138.1, 127.6, 71.2, 61.4, 57.9, 57.6, 29.5, 23.2, 14.1 ppm; MS (FAB) m/z (%): 269 (M+Hⁱ), 41 (44), 43 (100), 56 (54), 57 (46), 84 (22), 227 (55); HRMS calcd for C₁₁H₁₇N₄O₄ 269.1250; found 269.1248.

### Example 20

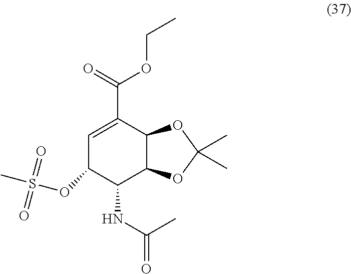

To a stirred solution of 32 (40 mg, 0.13 mmol) and triethylamine (37 μL, 0.27 mmol) in methylene chloride (100 μL) was added mesityl chloride (13 μL, 0.16 mmol) at 0° C. The reaction was slowly raised to room temperature over 12 h before extracting into EtOAc (5×1 mL). The combined organic layers were washed with sat. NH₄Cl (2×1 mL), brine (1×1 mL), and dried over Na₂SO₄. The crude material was purified via flash column chromatography with a solvent gradient of 1:1 then 1:3 (hexane-ethyl acetate) to yield 37 (45 mg, 90%) as a white solid: R_{f} 0.22 (1:5 hexanes-ethyl acetate); mp 163-165° C. (hexanes-ethyl acetate); [α]_{D}²³ −109.6 (c 1.25, CHCl₃); IR (film) ν 3307, 2628, 1719, 1651, 1361 cm⁻¹; ¹H NMR (300 MHz, CDCl₃) δ 6.98 (d, J=4.3 Hz, 1H), 5.68 (d, J=8.3 Hz, 1NH), 5.46 (t, J=4.1 Hz, 1H), 5.01 (d, J=5.3 Hz, 1H), 4.58 (td, J=4.0, 4.1 Hz, 1H), 4.34 (dd, J=5.5, 8.1 Hz, 1H), 4.24-4.32 (m, 2H), 3.07 (s, 3H), 2.05 (s, 3H), 1.43 (s, 3H), 1.42 (s, 3H), 1.34 (s, 3H) ppm; ¹³C NMR (75 MHz, CDCl₃) δ 170.8, 164.5, 134.7, 133.5, 110.7, 73.5, 73.0, 69.9, 61.7, 49.8, 38.7, 27.6, 25.9, 23.3, 14.1 ppm; MS (FAB) m/z (%): 378 (M+Hⁱ), 29 (17), 43 (39), 102 (100), 136 (24), 182 (19), 224 (18), 320 (53), 378 (41); HRMS calcd for C₁₅H₂₄NO₈S⁺378.1223; found 378.1195

### Example 21

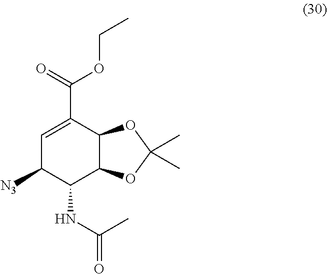

To a stirred solution of 37 (40 mg, 0.11 mmol) in 3:1-acetone: H₂O (1 mL) was added sodium azide (69 mg, 1.1 mmol) at room temperature. The resulting solution was stirred for 24 hrs, and then extracted with ethyl acetate (5×1 mL). The combined organic layers were washed with brine (1×1 mL) and then dried over Na₂SO₄. The crude material was recrystallized from hexanes-ethyl acetate to yield 30 (30 mg, 87%) as a pale yellow solid. R_{f} 0.24 (1:5 hexanes-ethyl acetate); mp 90° C. (hexanes-ethyl acetate); [α]_{D}²³+65.5 (c 1.15, CHCl₃); IR (film) ν 3298, 2983, 2103, 1722, 1658, 1372, 1251 cm⁻¹; ¹H NMR (600 MHz, CDCl₃) δ 6.91 (d, J=2.0 Hz, 1H), 6.14 (d, J=8.2 Hz, 1NH), 4.93 (d, J=5.6 Hz, 1H), 4.54 (dd, J=2.0, 10.1 Hz, 1H), 4.46 (ddd, J=1.7, 5.6, 10.1 Hz, 1H), 4.26 (q, J=7.1 Hz, 2H), 3.68 (dt, J=8.2, 9.6 Hz, 1H), 2.05 (s, 3H), 1.49 (s, 3H), 1.41 (s, 3H), 1.31 (s, 3H) ppm; ¹³C NMR (150 MHz, CDCl₃) δ 171.4, 164.5, 140.4, 129.3, 110.9, 74.1, 70.7, 61.5, 58.9, 54.3, 28.2, 26.1, 23.7, 14.1 ppm; MS (EI) m/z (%): 309 (M-CH₃⁺), 41 (44), 43 (100), 56 (54), 57 (46), 84 (22); HRMS calcd for C₁₃H₁₇N₄O₅ 309.1199; found 309.1200.

### Example 22

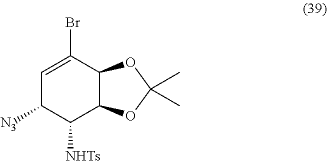

To a stirred solution of 38 (200 mg, 0.499 mmol) in DMF (2 mL) was added NH₄Cl (400 mg) and NaN₃ (324 mg, 4.99 mmol) at 0° C. The reaction mixture was stirred for 12 hrs, extracted into Et₂O (5×1 mL), washed with H₂O (20×0.5 mL), washed with brine (1×1 mL) and dried over Na₂SO₄. The crude material was recrystallized from CHCl₃-hexanes to yield 39 (203 mg, 92%) as a white solid: R_{f} 0.53 (2:1 hexanes-ethyl acetate); mp 119-120° C. (CHCl₃-hexanes); [α]_{D}²³+82.36 (c 3.5, CHCl₃); IR (film) ν 3435, 2108, 1645, 1599, 1219, 1159 cm⁻¹; ¹H NMR (600 MHz, CDCl₃) δ 7.80 (d, J=8.3 Hz, 1H), 7.31 (d, J=8.2 Hz, 2H), 6.14 (d, J=3.4 Hz, 1H), 5.61 (d, J=8.3 Hz, 1H), 4.58 (d, J=5.6 Hz, 1H), 4.19 (dd, J=5.8, 6.9 Hz, 1H), 3.73 (dd, J=3.0, 6.4 Hz, 1H), 3.56 (q, J=7.5 Hz, 1H), 2.45 (s, 3H), 1.37 (s, 3H), 1.34 (s, 3H) ppm; ¹³C NMR (150 MHz, CDCl₃) δ 143.6, 137.5, 129.7, 129.0, 127.3, 127.2, 127.1, 127.0, 124.0, 111.3, 75.9, 75.6, 60.5, 55.2, 27.5, 25.9, 21.5 ppm; MS (EI) m/z (%): 442 (M), 43 (47), 91 (100), 155 (35); HRMS calcd for C₁₅H₁₆N₄O₄BrS′. 442.0076; found 442.0072.

### Example 23

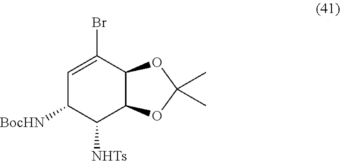

To a stirred solution of 39 (41 mg, 0.092 mmol) in 12:1 THF:H₂O (1 mL) was added triphenylphosphine (48 mg, 0.18 mmol). The reaction mixture was stirred for 12 hrs, extracted into Et₂O (5×0.5 mL), washed with brine (1×1 mL) and dried over Na₂SO₄. To a stirred solution of the crude Staudinger intermediate 40 (0.092 mmol) in DCM (1 mL) and triethylamine (50 μL) was added (Boc)₂O (29 mg, 0.14 mmol) at 0° C. The reaction mixture was stirred for 12 hrs, extracted into CHCl₃ (5×0.5 mL), washed with sat. NH₄Cl (2×1 mL), washed with brine (1×1 mL) and then dried over Na₂SO₄. The crude material was purified by flash column chromatography with a solvent gradient of 10:1, 6:1 then 3:1 (hexane-ethyl acetate) to yield 41 (36 mg, 77%): R_{f} 0.31 (2:1 hexanes-ethyl acetate); mp 153-154° C. (EtOAc-hexanes); [α]_{D}²³ +15.467 (c 2.1, CHCl₃); IR (film) ν 3408, 2090, 1642, 1161 cm⁻¹; ¹H NMR (600 MHz, CDCl₃) δ 7.77 (d, J=7.9 Hz, 1H), 7.27 (d, J=7.9 Hz, 2H), 6.17 (d, J=3.4 Hz, 1H), 5.32 (d, J=8.9 Hz, 1H), 5.26 (d, J=8.2 Hz, 1H), 4.60 (d, J=5.6 Hz, 1H), 4.23 (dd, J=6.2, 6.9 Hz, 1H), 4.08-4.13 (m, 1H), 3.46 (q, J=7.4 Hz, 1H), 2.40 (s, 3H), 1.43 (s, 9H), 1.29 (s, 3H), 1.18 (s, 3H) ppm; ¹³C NMR (150 MHz, CDCl₃) δ 155.6, 143.5, 137.5, 132.7, 129.6, 127.32, 121.4, 110.9, 80.3, 76.4, 76.3, 55.0, 51.2, 28.3, 27.3, 25.9, 21.5 ppm; MS (EI) m/z (%): 516 (M), 57 (46), 91 (81), 98 (62), 99 (97), 139 (48), 254 (100); HRMS calcd for C₂₁H₂₉N₂O₆SBr 516.0930; found 516.0930.0940.

### Example 24

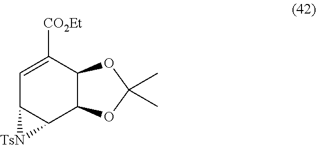

To a stirred solution of 23 (500 mg, 2.23 mmol) and Cu(acac)₂ (58 mg, 0.223 mmol) in MeCN (1 mL) was added PhI=NTs (832 mg, 2.23 mmol) at 0° C. The resulting solution was stirred for 5 h before filtering through SiO₂ and concentrating. The crude material was purified via flash column chromatography with a solvent gradient of 10:1, 6:1 then 3:1 (hexane-ethyl acetate) to yield 42 (359 mg, 41%) as colourless crystals: R_{f} 0.46 (2:1 hexanes-ethyl acetate); mp 106-107° C. (MeOH-hexanes); [α]_{D}²³ −52.526 (c 0.62, CHCl₃); IR (film) ν 3434, 2099, 1647, 1160 cm⁻¹; ¹H NMR (600 MHz, (CO(CD₃)₂) δ 7.86 (d, J=8.3 Hz, 2H), 7.48 (d, J=8.3 Hz, 2H), 6.97 (d, J=4.5 Hz, 1H), 4.73 (d, J=6.9 Hz, 1H), 4.62 (dd, J=0.9, 6.9 Hz, 1H), 4.14-4.21 (m, 2H), 3.56 (dd, J=4.5, 6.2 Hz, 1H), 3.34 (dd, J=1.1, 6.4 Hz, 1H), 2.45 (s, 3H), 1.32 (s, 3H), 1.31 (s, 3H), 1.23 (t, J=7.1 Hz, 3H) ppm; ¹³C NMR (150 MHz, CDCl₃) δ 164.6, 145.2, 134.7, 134.1, 131.2, 130.0 (2×C), 127.9 (2×C), 110.3, 70.0, 68.6, 60.7, 37.5, 35.3, 27.0, 24.9, 20.8, 13.6 ppm; MS (EI) m/z (%): 393 (M), 43 (38), 47 (29), 49 (24), 47 (100), 86 (80), 91 (20); HRMS calcd for C₁₉H₂₃NO₆S 393.1246; found 393.1239.

### Example 25

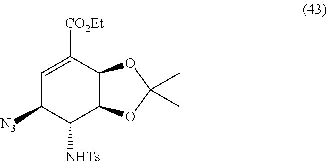

To a stirred solution of 42 (25 mg, 0.0636 mmol) and NH₄Cl (51 mg, 0.953 mmol) in DMF (0.5 mL) was added NaN₃ (8 mg, 0.127 mmol) at 0° C. The resulting suspension was stirred for 3 h before diluting with Et₂O (1 mL) and H₂O (1 mL). The layers were separated and the aqueous layer was extracted with Et₂O (10×0.2 mL). The combined organic layers were washed with brine (1×1 mL) and dried over Na₂SO₄. The crude material was purified via flash column chromatography with a solvent gradient of 4:1 then 2:1 (hexane-ethyl acetate) to yield 43 (22 mg, 79%) as a white solid: R_{f} 0.43 (1:1 hexanes-ethyl acetate); ¹H NMR (300 MHz, CDCl₃) δ 7.83 (d, J=8.3 Hz, 2H), 7.30 (d, J=8.3 Hz, 2H), 6.87 (d, J=2.6 Hz, 1H), 4.99 (d, J=7.8 Hz, 1H), 4.91 (d, J=5.5 Hz, 1H), 4.26 (q, J=7.1 Hz, 2H), 4.10 (dd, J=5.7, 9.1 Hz, 1H), 3.93 (dd, J=2.6, 9.0 Hz, 1H), 3.45-3.54 (m, 1H), 2.42 (s, 3H), 1.32-1.37 (m, 4H), 1.28-1.32 (m, 5H) ppm; MS (FAB) m/z (%): 437 (M+H^{f}), 43 (18), 91 (100), 136 (20), 139 (39), 152 (23), 155 (72), 167 (24), 168 (22), 181 (27), 196 (38), 437 (27); HRMS calcd for C₁₉H₂₅N₄O₆S 437.1495; found 437.1494

### Example 26

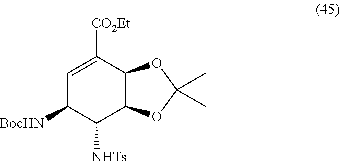

A solution of 41 (2 g, 3.86 mmol), ethanol (15 mL) and triethylamine (15 mL) in toluene (70 mL) was purged with CO (g) for 10 mins. The solution was charged with CO(g), Pd(PPh₃)₄ (223 mg, 0.193 mmol) and then heated to 60° C. for 1 hr before the addition of Pd[(PPh₃)₂ (Cl)₂] (271 mg, 0.386 mmol). The reaction was then heated to reflux for 6 hrs, cooled to r.t. and filtered through a plug of SiO₂. The crude material was purified by flash column chromatography to yield 45 (888 mg, 45%) as a yellow oil: R_{f} 0.46 (1:1 hexanes-ethyl acetate); [α]_{D}²³ −11.77 (c 0.72, CHCl₃); IR (film) ν 3434, 2099, 1647, 1160 cm⁻¹; ¹H NMR (300 MHz, CDCl₃) δ 7.78 (d, J=8.2 Hz, 2H), 7.26 (d, J=8.2 Hz, 2H), 6.89 (d, J=2.2 Hz, 1H), 5.36 (d, J=8.2 Hz, 1H), 5.23 (d, J=8.3 Hz, 1H), 4.92 (d, J=5.6 Hz, 1H), 4.23 (q, J=7.0 Hz, 2H), 4.10 (dd, J=6.0, 8.9 Hz, 1H), 3.38 (q, J=8.7 Hz, 1H), 2.39 (s, 3H), 1.46 (s, 9H), 1.29 (t, J=7.1 Hz, 3H), 1.26 (s, 3H), 1.05 (s, 3H) ppm; ¹³C NMR (75 MHz, CDCl₃) δ 171.1, 143.5, 136.7, 129.6, 127.6, 109.5, 78.3, 74.0, 60.7, 42.9, 28.7, 27.4, 26.0, 21.5, 20.5, 14.1 ppm; MS (EI) m/z (%): 510 (M), 43 (100), 57 (52), 84 (54); HRMS calcd for C₂₄H₃₄N₂O₈S 510.2036; found 510.2038.

### Example 27

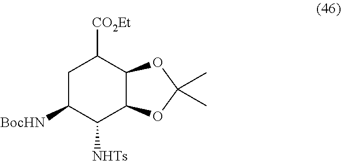

A hydrogenation vial was charged with 45 (240 mg, 0.470 mmol), and 5% Rh/Al₂O₃ (60 mg) and 85% ethanol (1.5 mL) before evacuating with H₂. The reaction was stirred at room temperature and 55 psi for 144 h before filtering through a plug of SiO₂ and concentrating. The crude material was purified via flash column chromatography with a solvent gradient of 3:1 then 1:1 (hexanes-ethyl acetate) to yield 46 (228 mg, 95%) as a white solid: R_{f} 0.49 (1:1 hexanes-ethyl acetate); mp 246-247° C. (CHCl₃); IR (film) ν 3434, 2099, 1647, 1160 cm⁻¹; ¹H NMR (600 MHz, CDCl₃) δ 7.78 (d, J=8.3 Hz, 2H), 7.24 (d, J=8.3 Hz, 2H), 5.30 (d, J=8.6 Hz, 1H), 5.12 (d, J=8.3 Hz, 1H), 4.46 (t, J=4.3 Hz, 1H), 4.18-4.25 (m, 1H), 4.09-4.15 (m, 1H), 3.86 (dd, J=5.1, 8.8 Hz, 1H), 3.40 (dq, J=1.3, 10.2 Hz, 1H), 3.21 (q, J=9.3 Hz, 1H), 2.81 (dt, J=3.8, 12.6 Hz, 1H), 2.42 (s, 3H), 2.13 (dt, J=3.7, 13.5 Hz, 1H), 1.80 (q, J=12.8

Hz, 1H), 1.45 (s, 9H), 1.22 (t, J=6.9 Hz, 3H), 1.18 (s, 3H), 1.16 (s, 3H) ppm; ¹³C NMR (150 MHz, CDCl₃) δ 170.3, 156.4, 142.8, 138.6, 129.1, 127.4, 109.4, 80.1, 79.2, 73.8, 60.9, 60.2, 50.4, 41.2, 28.5, 28.4, 27.7, 26.0, 21.4, 14.1 ppm; MS (EI) m/z (%): 497 (M-CH₃), 41 (52), 43 (44), 57 (100), 91 (99), 100 (35), 155 (44), 182 (46), 240 (47), 257 (79); HRMS calcd for C₂₃H₃₃N₂O₈S 497.1958, found 497.1962.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A process for the preparation of oseltamivir and analogs thereof of the formula I: 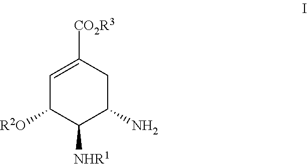
wherein R¹ is C₁₋₆acyl;
R² is C₁₋₆alkyl; and
R³ is C₁₋₆alkyl,
said process comprising:
(i) converting a halobenzene of the formula II, wherein X is halo, to the diol of the formula III, wherein X is halo, by toluene dioxygenase mediated oxidation, 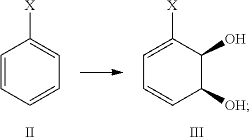
(ii) protecting the hydroxyl groups of the diol of formula III, followed by aziridination to provide a compound of the formula IV, wherein X is halo, R¹ is C₁₋₆acyl and PG¹ and PG² are suitable protecting groups that are optionally linked to form, together with the oxygen atoms to which they are attached, an optionally C₁₋₆alkyl-substituted 5- or 6-membered ring, 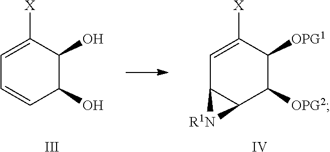
(iii) opening the aziridine ring in the compound of the formula IV with an alcohol of the formula R²—OH, wherein R² is C₁₋₆alkyl, followed by palladium catalyzed carbonylation to provide a compound of the formula V, wherein R¹ is C₁₋₆acyl, R² is C₁₋₆alkyl, R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, 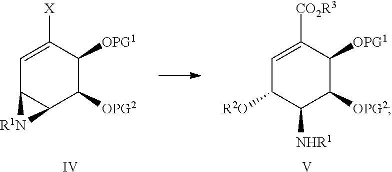
(iv) deprotecting the compound of the formula V and selectively converting the hydroxyl adjacent to the NHR¹ group to a leaving group (LG) to provide a compound of the formula VI, wherein R¹ is C₁₋₆acyl, R² is C₁₋₆alkyl, R³ is C₁₋₆alkyl and LG is a suitable leaving group, 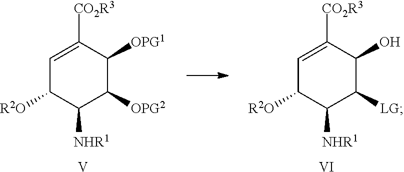
(v) displacing the leaving group in the compound of formula VI with an azide group to provide a compound of the formula VII, wherein R¹ is C₁₋₆acyl, R² is C₁₋₆alkyl and R³ is C₁₋₆alkyl, 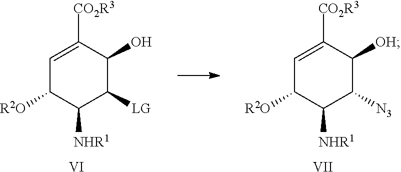
and
(vi) conversion of the hydroxyl group in the compound of the formula VII to a group removable by reduction and treating the resulting compound under reductive conditions to remove the group and converting the azide group to an amino group to provide a compound of formula I, 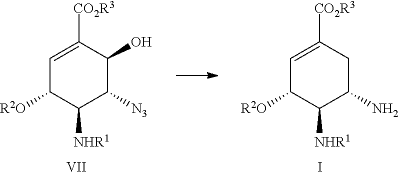
wherein, in the compounds of the formulae I-VII, one or more hydrogens in R¹, R² and/or R³ is/are optionally replaced with F.

2. The process according to claim 1, wherein the compound of formula I is converted to a pharmaceutically acceptable salt.

3. The process according to claim 2, wherein the pharmaceutically acceptable salt is a H₃PO₄ salt.

4. A process for the preparation of oseltamivir and analogs thereof of the formula I: 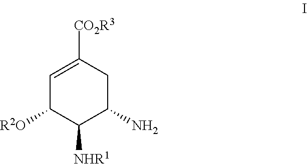
wherein R¹ is C₁₋₆acyl;
R² is C₁₋₆alkyl; and
R³ is C₁₋₆alkyl,
said process comprising:
(i) protecting the hydroxyl groups of a compound of formula III, wherein X is halo, as the di(C₁₋₆)alkyl ketal followed by aziridination to provide a compound of the formula VIII, wherein X is halo, R⁴ and R⁵ are independently, C₁₋₆alkyl and R⁶ is C₁₋₆acyl, 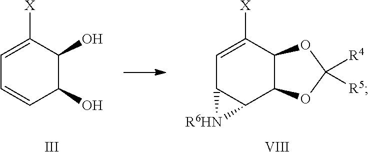
(ii) ring opening the aziridine ring of the compound of formula VIII with PG³-NH₂, where PG³ is a suitable protecting group, followed by palladium catalyzed carbonylation to provide a compound of the formula IX, wherein R³, R⁴ and R⁵ are, independently, C₁₋₆alkyl, R⁶ is C₁₋₆acyl and PG³ is a suitable protecting group, 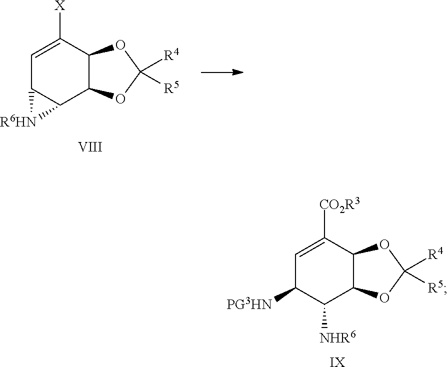
(iii) regioselective ring-opening of the cyclic ketal in the compound of formula IX and reduction to provide a compound of the formula X, wherein R³, R⁴ and R⁵ are, independently, C₁₋₆alkyl, R⁶ is C₁₋₆acyl and PG³ is a suitable protecting group, 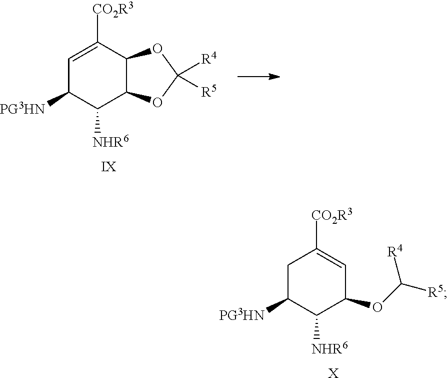
or, removing the cyclic ketal in the compound of formula IX followed by rearranging and eliminating one hydroxy group to provide a compound of the formula XI, wherein R³ is C₁₋₆alkyl, R⁶ is C₁₋₆acyl and PG³ is a suitable protecting group followed by alkylating the remaining hydroxy group with a compound of the formula R²-LG, wherein R² is C₁₋₆alkyl and LG is a suitable leaving group, to provide a compound of the formula XII, wherein R³ is C₁₋₆alkyl, R⁶ is C₁₋₆acyl and PG³ is a suitable protecting group, 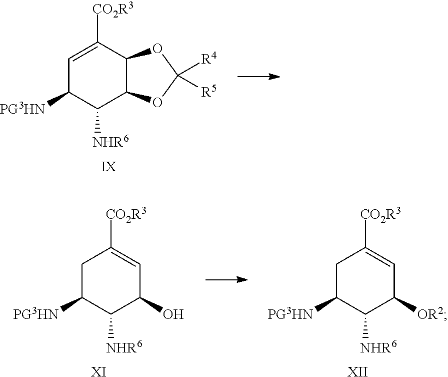
(iv) removing the PG³ of the compound of the formula X or XII to provide a compound of the formula I, 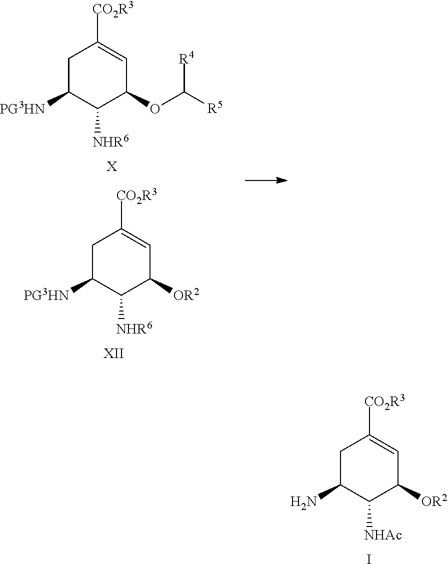
wherein, in the compounds of the formulae I, III and VIII-XII, one or more hydrogens in R¹, R², R³, R⁴, R⁵, and/or R⁶ is/are optionally replaced with F.

5. The process according to claim 4, wherein the compound of formula I is converted to a pharmaceutically acceptable salt.

6. The process according to claim 5, wherein the pharmaceutically acceptable salt is a H₃PO₄ salt.

7. A process for the preparation of compounds of formula IX 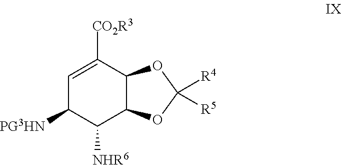
wherein R³, R⁴ and R⁵ are, independently, C₁₋₆alkyl, R⁶ is C₁₋₆acyl and PG³ is a suitable protecting group, said process comprising:
(i) converting a benzoate ester of formula XIII, wherein R³ is C₁₋₆alkyl, to a compound of the formula XIV, wherein R³ is C₁₋₆alkyl, by toluene dioxygenase mediated oxidation, 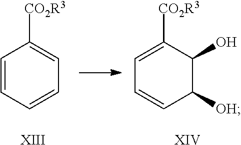
(ii) protecting the hydroxyl groups of the compound of formula XIV as the di(C₁₋₆alkyl) ketal followed by regio- and stereoselective nitroso Diels-Alder cycloaddition to the conjugated diene to provide a compound of the formula XV, wherein R³, R⁴ and R⁵ are, independently, C₁₋₆alkyl and R⁶ is C₁₋₆acyl, 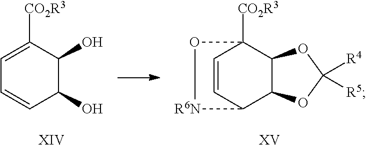
and
(iii) displacing the oxazine bridge of the compound of formula XV with PG³NH₂, wherein PG³ is a suitable protecting group, under palladium catalyzed conditions followed by reduction to provide a compound of the formula IX, wherein R³, R⁴ and R⁵ are, independently, C₁₋₆alkyl and R⁶ is C₁₋₆acyl and PG³ is a suitable protecting group, 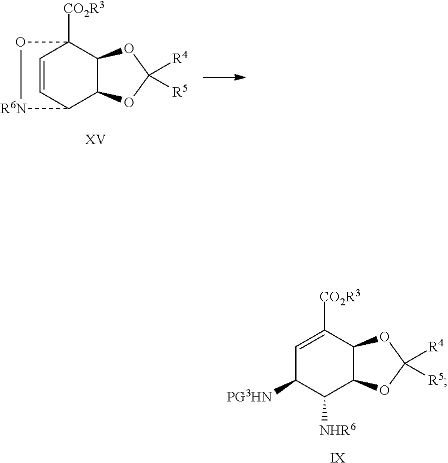
or,
ring opening of the oxazine bridge of the compound of formula XV to yield the compound of formula XVI, followed by converting the hydroxy group in the compound of formula XVI to a leaving group and allylic displacement of the leaving group with PG³NH₂ or azide, wherein PG³ is a suitable protecting group, to provide, after reduction of the azide and protection of the resulting amine with PG³, a compound of the formula IX, wherein R³, R⁴ and R⁵ are, independently, C₁₋₆alkyl and R⁶ is C₁₋₆acyl and PG³ is a suitable protecting group 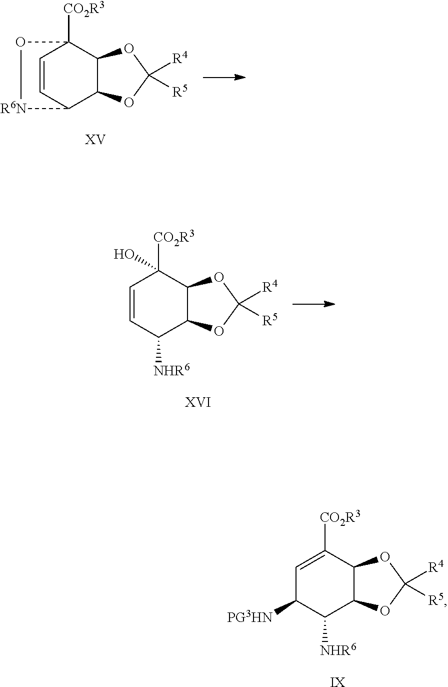
wherein, in the compounds of the formulae IX and XIII-XVI, one or more hydrogens in R³, R⁴, R⁵, and/or R⁶ is/are optionally replaced with F.

8. A process of preparing a compound of the formula XVIII 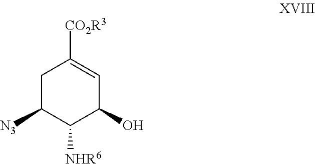
wherein R³ is C₁₋₆alkyl and R⁶ is C₁₋₆acyl, said process comprising:
(i) converting the hydroxyl group of a compound of the formula XVI, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, into a suitable leaving group in the presence of a base to provide a compound of formula XIX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, 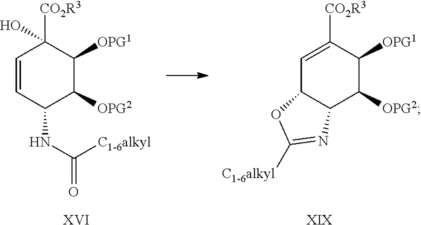
(ii) cleaving the oxazoline ring of the compound of formula XIX, followed by hydrogenation to provide a compound of the formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, 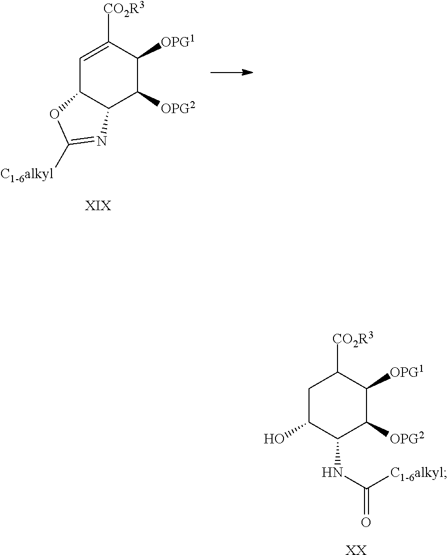
and
(iii) converting the hydroxyl group of the compound of formula XX to a suitable leaving group, displacing the leaving group with azide and treating the resulting intermediate with a suitable base to provide a compound of the formula XVIII, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, 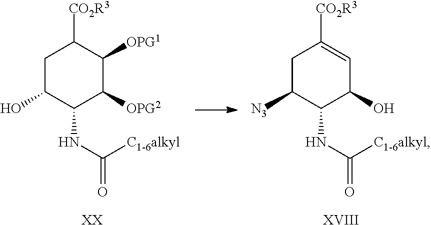
wherein, in the compounds of formulae XVI, XIX, XX and XVIII, one or more hydrogens is R³ and/or R⁶ is/are optionally replaced with F.

9. A process for the preparation of compounds of formula XXI, wherein R³ is C₁₋₆alkyl and PG³ is a suitable protecting group 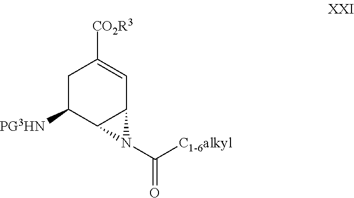
the process comprising:
(i) converting the hydroxyl group of a compound of formula XX, wherein R³ is C₁₋₆alkyl and PG¹ and PG² are suitable protecting groups that are optionally linked to form, together with the oxygen atoms to which they are attached, an optionally C₁₋₆alkyl-substituted 5- or 6-membered ring, to a suitable leaving group, displacing the leaving group with a compound of the formula PG³NH₂ wherein PG³ is a suitable leaving group to provide a compound of the formula XXII, wherein R³ is C₁₋₆alkyl and PG¹, PG² and PG³ are suitable protecting groups and PG¹ and PG² are optionally linked to form, together with the oxygen atoms to which they are attached, an optionally C₁₋₆alkyl-substituted 5- or 6-membered ring, 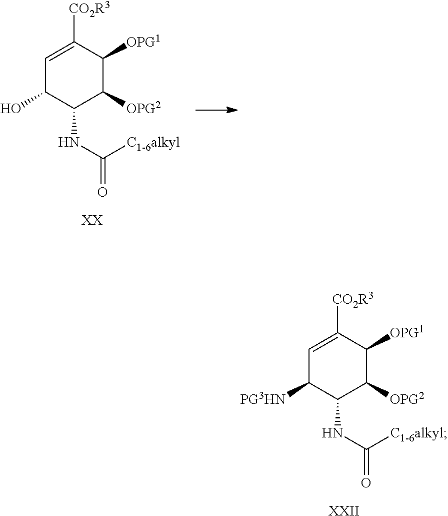
(ii) reducing double bond in the ring of the compound of formula XXII followed by treating the resulting saturated ring compound with a suitable base to provide a compound of the formula XI, wherein R³ is C₁₋₆alkyl and PG³ is a suitable protecting group, 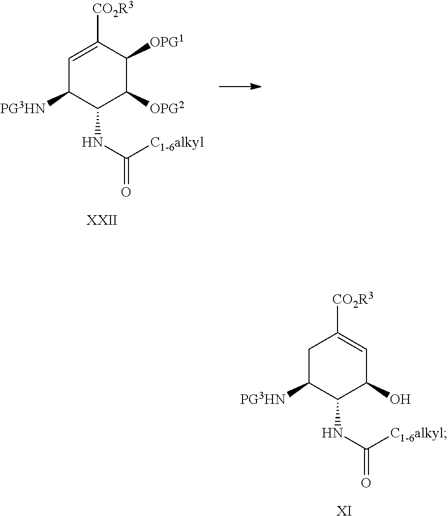
and
(iii) converting the hydroxyl group in the compound of formula XI to a leaving group and treating the resulting compound with a suitable base to provide a compound of the formula XXI, wherein R³ is C₁₋₆alkyl and PG³ is a suitable protecting group 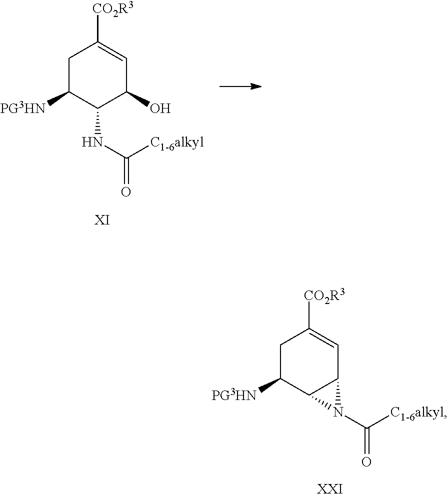
wherein in the compounds of the formulae XX, XXI, XXII and XI, one or more of the hydrogens is/are optionally replaced with F.

10. The process according to claim 1, wherein X is Br.

11. The process according to claim 1, wherein PG¹ and PG² are linked for form a dimethyl or diethyl ketal protecting group.

12. The process according to claim 1, wherein R¹ is acetyl.

13. The process according to claim 1, wherein R² is 3-pentyl.

14. The process according to claim 1, wherein R³ is ethyl.

15. A compound of the formula A: 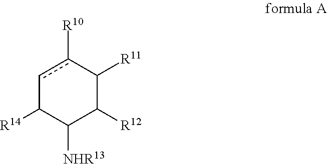
wherein,
R¹⁰ is selected from halo and CO₂R¹⁵;
R¹¹ is selected from OH and OR¹⁶;
R¹² is selected from OH, OR¹⁷ and N₃;
R¹³ is selected from H and C₁₋₆acyl;
R¹⁴ is selected from OC₁₋₆alkyl, SC₁₋₆alkyl, OH, SH, halo, N₃, NH₂, NHC₁₋₆alkyl and NHPG⁴, or
R¹³ and R¹⁴ are linked to form, together with the atoms to which they are attached, and oxazoline ring;
R¹⁵ is C₁₋₆alkyl;
R¹⁶ and R¹⁷ are the same or different and are, independently, PG⁵ or R¹⁶ and R¹⁷ are joined together, to form, together with the oxygen atoms to which they are attached, a 5-membered cyclic ketal that is substituted on the carbon between the oxygen atoms by one or two C₁₋₆alkyl;
PG⁴ and PG⁵ are, independently protecting groups; [symbol]
represents a single or double bond, and
one or more hydrogens in the C₁₋₆alkyl and/or C₁₋₆acyl groups is/are is optionally replaced with F,
or salts, solvates, prodrugs, stereoisomers or isotope-labelled forms thereof, or mixtures thereof,
provided that when R¹⁴ is NHPG⁴ or NHC₁₋₆alkyl, R¹⁰ is CO₂Et, R¹¹ is OH or OPG⁴ and PG⁴ is acyl, then R¹² is not 3-pentoxy.

16. A compound of formula B: 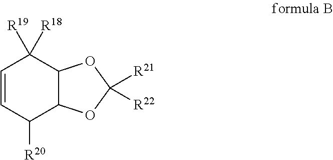
wherein,
R¹⁸ is CO₂R²⁴;
R¹⁹ is selected from H, OH and OC₁₋₆acyl;
R²⁰ is NHC₁₋₆acyl;
or the O in R¹⁹ and the N in R²⁰ are joined by a covalent bond;
R²¹ and R²² are, independently, C₁₋₆alkyl;
R²⁴ is C₁₋₆alkyl, and
one or more of the hydrogens in C₁₋₆alkyl and C₁₋₆acyl is/are optionally replaced with F, or salts, solvates, prodrugs, stereoisomers or isotope-labelled forms thereof, or mixtures thereof.

17. A compound selected from: 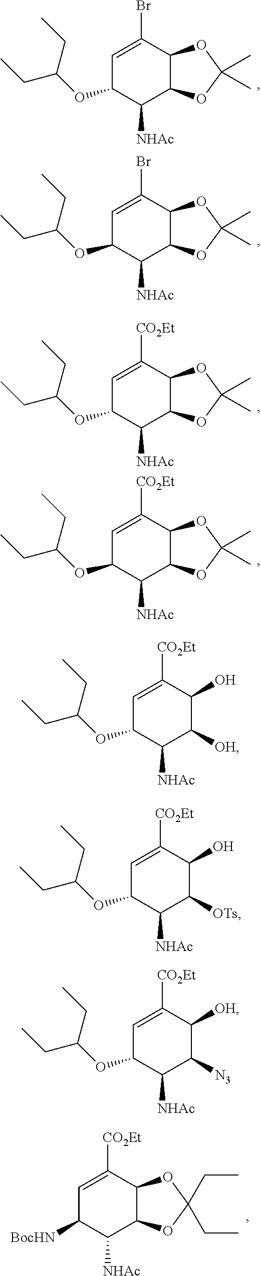 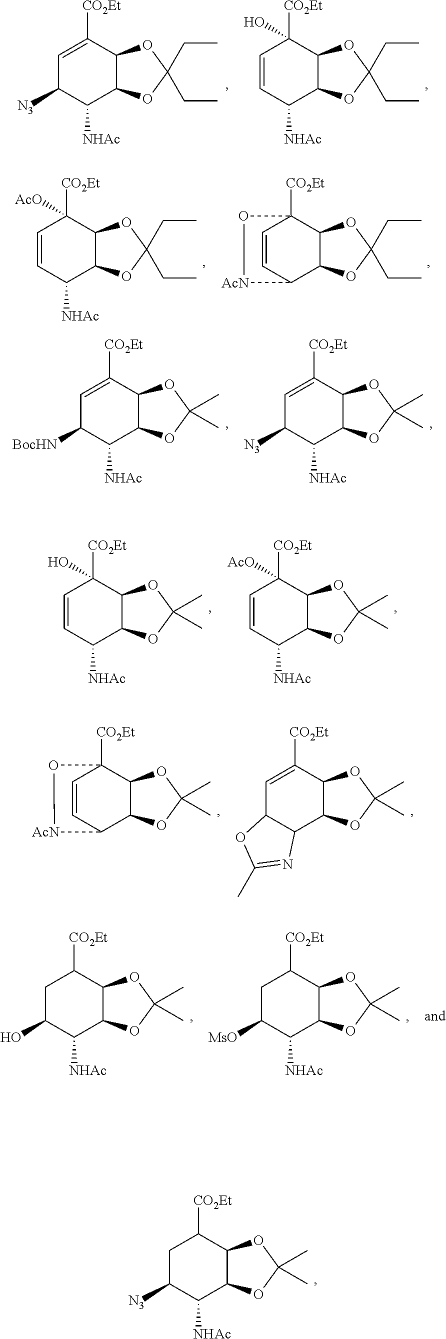
or salts, solvates, prodrugs, stereoisomers or isotope-labelled forms thereof, or mixtures thereof.

18. A pharmaceutical composition comprising a compound according to claim 15 and a pharmaceutically acceptable carrier or diluent.

19. A method of treating or preventing influenza comprising administering an effective amount of a compound according to claim 15 to a subject in need thereof.

20. A method of treating or preventing influenza comprising administering an effective amount of compound according to claim 17 to a subject in need thereof.
